(19) United States
(12) Patent Application Publication
(10) Pub. No.: US 2011/0052627 A1
(43) Pub. Date: Mar. 3, 2011

(54) RECOMBINANT MODIFIED VACCINIA VIRUS MEASLES VACCINE

(76) Inventor: Paul Chaplin, Munich (DE)

(21) Appl. No.: 12/937,412

(22) PCT Filed: Jun. 8, 2009
(86) PCT No.: PCT/EP09/04094

§ 371 Date: Oct. 12, 2010

Related U.S. Application Data
(60) Provisional application No. 61/074,406, filed on Jun. 20, 2008.

(30) Foreign Application Priority Data
Jul. 1, 2008 (EP) 08011868.0

(51) Int. Cl. A61K 39/165; C12N 7/04; C12N 5/071; A61P 31/14; A61P 37/04

(52) U.S. Cl. 424/199.1; 435/236; 435/325

(57) ABSTRACT

The invention concerns methods, compositions and kits for use in preparing a medicament and vaccine for measles virus comprising an Attenuated Modified Vaccinia Virus Ankara (MVA) strain encoding hemagglutinin protein, fusion protein, and nucleoprotein of measles virus (MVA-Measles). The recombinant virus induced superior cellular and humoral responses to the measles virus when compared to Measles vaccine Rouvax®. Both T cell and B cell immune responses to the recombinant MVA were observed not only in adult animals, but also in newborn and juvenile animals. Results in adult humans showed that MVA-Measles induces a strong immune response, is safe and well tolerated.

The present invention relates to a recombinant Modified Vaccinia virus Ankara (MVA) comprising in its genome the hemagglutinin (H), fusion (F), and nucleoprotein (N) gene of measles virus and/or an antigenic epitope of one, two or all of said measles virus antigens. The invention also relates to a pharmaceutical composition, a vaccine and a kit including said recombinant MVA virus. The invention further encompasses the use of the recombinant virus for immunizing an animal body, including a human, against measles virus infection. The invention further relates to a method of generating the recombinant MVA, a method of producing measles virus antigens and/or epitopes, and to a method of introducing said antigens and/or epitopes into a cell. Also encompassed by the present invention is a cell comprising the recombinant MVA.

### BACKGROUND OF THE INVENTION

Although measles is now rare in industrialized countries, it remains a common illness in other parts of the world. More than 20 million people are affected each year. In 2005, it was estimated 345,000 individuals died of measles globally, the majority of them children younger than 5 years (Wolfson et al., 2007, Has the 2005 measles mortality reduction goal been achieved? A natural history modelling study. Lancet 369: 191-200). Measles is one of the most contagious diseases known. People who recover from measles are immune for the rest of their lives (WHO Fact Sheet N° 286, Revised 2007).

Almost all non-immune children contract measles if exposed to the virus. Malnourished young children or immunocompromised children are particularly at risk of developing severe forms of the infection. The most serious complications include blindness, encephalitis, severe diarrhea, ear infections and severe respiratory infections such as pneumonia, which is the most common cause of death associated with measles. Encephalitis is estimated to occur in 1/1000 infected subjects, otitis media in 1/20-1/7 and pneumonia in 1/20-1/10. The case fatality rate in developing countries is generally in the range of 1/100-1/20, but may be as high as 25% in populations with high levels of malnutrition and poor access to health care (WHO Fact Sheet N° 286, Revised 2007). Another severe sequelae of measles virus infection is a syndrome called subacute sclerosing panencephalitis (SSPE), a fatal disease of the central nervous system that generally develops 7-10 years after infection and leads to death within 1-3 years. It is induced by a persistent defective measles virus with an incidence of 4-11 cases per 100,000 subjects with a measles infection but may be higher (18 per 100,000 cases) when measles is acquired very early in life (WHO Weekly Epidemiological Record. 13 Jan. 2006). Epidemiological data show a directly protective effect of measles vaccination against SSPE (Garg, 2002, Subacute sclerosing panencephalitis. Postgrad Med J. 78: 63-70; WHO Weekly Epidemiological Record. 13 Jan. 2006; Bellini et al., 2005, Subacute sclerosing panencephalitis: more cases of this fatal disease are prevented by measles immunization than was previously recognized. J Infect Dis. 192(10): 1686-93). The table below provides an overview of the incidence of complications after natural measles infection and measles vaccination with MMR, a vaccine also directed against mumps and rubella.

TABLE 1
Incidence of Complications After Natural Measles Infection or
Vaccination

| | | Measles Vaccination |
|---|---|---|
| Complication | Measles Disease | (MMR) |
| Fever and all | | 1/50-1/7 |
| Febrile | 1/200 | 1/3000 |
| Pneumonia | 1/20-1/10 | |
| Ear infection | 1/20 | |
| Otitis media | 1/20-1/7 | |
| Blindness | 1/100,000 | |
| Diarrhoea | 1/12 | |
| Conjunctivitis | | 1/50 |
| Anaphylaxis | | 1/1 million-1/20,000 |
| SSPE | 1/25,000-1/9,000 | none |
| Encephalitis | 1/1,000 | 1/3 million-1/100,000 |
| Lethality | 1/100-1/20 | |

MMR: Mumps - measles - rubella vaccine; Stephenson (2002), adapted to more recent report if available (WHO Fact Sheet N^{o}286, Revised 2007; WHO Weekly Epidemiological Record. 13 Jan. 2006; Bellini, 2005).

Live attenuated measles vaccines have successfully been used since 1963. They have contributed to a significant reduction in global cases of measles and have greatly diminished measles morbidity in the industrialized world. Global measles mortality decreased by more than 70% from 873,000 to 242,000 deaths between 1999 and 2006. The largest gains have occurred in Africa, where measles cases and deaths decreased by nearly 75% (Wolfson et al., 2007; WHO Fact Sheet N° 286, Revised 2007).

However, in a number of countries measles vaccination has proved less effective and as a result measles continues to be endemic. An important factor is that in these countries measles frequently affects children below the age of nine months, an age group particularly susceptible to severe measles infections and known to respond insufficiently to vaccination. (Stephenson, 2002, Will the current measles vaccines ever eradicate measles? Expert Rev Vaccines 1(3): 355-62). With the currently used measles vaccines, primary vaccine failure (the vaccine is not able to induce a protective immune response) is most common in younger infants (Kumar et al., 1998, Immune response to measles vaccine in 6-month-old infants of measles seronegative mothers. Vaccine 16(20): 2047-51. Erratum in: Vaccine 1999, 17(17): 2206; Gans et al., 2001, Immune responses to measles and mumps vaccination of infants at 6, 9, and 12 months. J Infect Dis. 184(7): 817-26), with a reduced seroconversion rate of 85% for vaccination at 9-11 months, compared to a seroconversion rate of 97% at 12-14 months or 100% at 15-17 months (Desgrandchamps et al., 2000, Seroprevalence of IgG antibodies against measles, mumps and rubella in Swiss children during the first 16 months of life. Schweiz Med. Wochenschr. 130(41): 1479-86). Below 9 months, the seroconversion rates are even lower.

The results of a study performed in Switzerland (Desgrandchamps et al., 2000) answered the question of how long maternal IgG antibodies against measles, mumps and rubella persist in infants. The following seroprevalence rates for IgG antibodies were found in the following order measles/mumps/rubella:
0-3 months: 97%/62%/91%.
>3-6 months: 40%/2%/42%.
>6-9 months: 4%/2%/10%.
>9-12 months: 2%/0%/12%.
>12-16 months: 0%/7%/7%.

These results demonstrate high levels of passive immunity against measles and rubella in Swiss infants during the first months of life. Beyond 12 months of age, IgG antibodies are only rarely detectable. Considering the current vaccination recommendations in developed countries to administer the first dose at 12-15 months, a gap of at least 6 to 9 months exists with reduced protection against a clinically relevant measles infection. (Maldonado et al., 1995, Early loss of passive measles antibody in infants of mothers with vaccine-induced immunity. Pediatrics (3 Pt 1): 447-50; Markowitz et al., 1996, Changing levels of measles antibody titers in women and children in the United States: impact on response to vaccination. Kaiser Permanente Measles Vaccine Trial Team. Pediatrics 97(1): 53-8; Desgrandchamps et al., 2000).

Additionally, sometimes the transfer of maternal antibodies is not sufficient and babies are susceptible to measles infections. In the developing countries the first measles vaccination is administered at 9 months of age to reduce the gap of limited protection in a still endemic environment, but in this age group the efficacy of the current measles vaccine is sub-optimal with regard to seroconversion rates and its ability to elicit sufficiently high titers for protective immunity.

In a study by Gans et al., 2001, the percentage of subjects with IgG antibodies using a plaque reduction neutralization test (PRNT), which is more sensitive compared to the ELISA assay, were:
At 6 months: 64%.
At 9 months: 39%.
At 12 months: 2%.

The PRNT is the accepted standard method for determining protective antibody levels against natural measles infection. The following table summarizes the geometric mean titers (GMTs) using the PRNT after vaccination with Attenuvax®, a monovalent, approved and widely-used measles vaccine that had been given to children at the age of 6 and 9 months, or with MMR-II®, an approved and widely-used combination vaccine against measles, mumps and rubella administered at 12 months.

TABLE 2
Geometric mean titers (GMTs) after vaccination with
Attenuvax ® and MMR-II ®

| | | | | | | GMTs | | |
|---|---|---|---|---|---|---|---|---|
| | | GMTs³ | 95% | 95% | | w/o | 95% | 95% |
| Age | n | w/ IgG¹ | CI LL² | CI UL² | n | IgG* | CI LL | CI UL |
| 6 m | 47 | 120 | 71 | 200 | 26 | 146 | 44 | 490 |
| 9 m | 24 | 180 | 68 | 473 | 37 | 744 | 467 | 1183 |
| 12 m | n.a | n.a. | n.a. | n.a. | 53 | 1210 | 774 | 1893 |

¹Determination of the presence of maternal antibodies, w = with, w/o = without;
²CI LL = confidence interval lower or upper limit
³GMT = geometric mean titer,

Only 59% of 6-month-old infants developed titers of ≧120 mIU/ml compared with 97% for 9- and 94% for 12-month-olds, who had no maternal antibodies. Cellular immune responses between these age groups were similar and comparable to adult subjects. Additionally, the current measles vaccine has significant problems with stability under varying conditions of temperature and light.

In another study, serum specimens were obtained from children before and 1 month after the first measles vaccine (Rouvax, Schwartz strain 1000 TCID₅₀) given at 9 months. A second dose was given to 72 children at 15 months of age as measles-mumps-rubella (Trimovax, Schwarz measles strain, 1000 TCID₅₀; Urabe Am 9 mumps strain, 5000 TCID₅₀; Wister RA 27/3 rubella strain, 1000 TCID₅₀). Third blood samples were collected 20 months after the second vaccine. The antibody positivity rate was 5.2% at the age of 9 months. Seroconversion rate was 77.6% after the first dose and 81.9% after the second dose of measles vaccine. Of 15 children who were seronegative, 13 (86.7%) became seropositive after the immunization at 15 months. Eleven children (19.2%) seroconverted from positive to negative after the second vaccine. (Isik et al., 2003, Pediatric Infectious Disease Journal. 22(8): 691-695).

The following categorization of Measles IgG antibody levels using the gold standard for measurement, the PRNT, are commonly used and accepted:
Negative <8 mIU/ml susceptible to infection and disease.
Low 8-120 mIU/ml potentially susceptible to infection and disease.
Medium 121-900 mIU/ml potentially susceptible to infection but not disease.
High >900 mIU/ml neither susceptible to infection nor disease.

It is accepted in the scientific and medical community that a titer above 120 mIU/ml will prevent any severe disease symptoms (Chen et al., 1990, Measles antibody: reevaluation of protective titers. J Infect Dis. 162(5): 1036-42; LeBaron et al., 2007, Persistence of measles antibodies after 2 doses of measles vaccine in a postelimination environment. Arch Pediatr Adolesc Med. 161(3): 294-301; Plotkin, 2001, Immunologic correlates of protection induced by vaccination. Pediatr Infect Dis J. 20(1): 63-75; Samb et al., 1995, Serologic status and measles attack rates among vaccinated and unvaccinated children in rural Senegal. Pediatr Infect Dis J. 14(3): 203-209).

However, the problem with the standard vaccine is obvious, considering that live measles vaccines do not work effectively in infants below 9 months of age. The difference between the two age groups can be explained by the presence of the maternal IgG antibodies and the inability of the infant to robustly produce immunoglobulin in response to vaccination. In the developing world, the highest morbidity or even mortality caused by measles infections occurs in infants below 6 months and the symptoms are more severe the younger the children are (Papania et al., 1999, Increased susceptibility to measles in infants in the United States. Pediatrics 104(5): e59; Hutchins et al., 1996, Measles outbreaks in the United States, 1987 through 1990. Pediatr Infect Dis J. 15(1): 31-8; Clements & Cutts, 1995, The epidemiology of measles: thirty years of vaccination. Curr Top Microbiol Immunol. 191: 13-33; Aaby and Clements, 1989, Measles immunization research: a review. Bull World Health Organ 67(4): 443-8). In other words, although the current vaccines are administered at 9 months, the seroconversion rate at this age is still not optimal. Another potential obstacle to the successful immunisation of younger infants includes the immaturity of their immune system (Kumar et al. 1998; Gans et al., 1998, Deficiency of the humoral immune response to measles vaccine in infants immunized at age 6 months. JAMA 280(6): 527-532).

Due to the limitations of the currently used live attenuated measles vaccines, new measles vaccines overcoming these shortcomings, especially the induction of a robust humoral and cellular immunity in very young children, could play an important role in the further reduction of measles morbidity and mortality and finally global eradication of the measles virus (CDC, 1998, Progress toward global measles control and regional elimination, 1990-1997. MMWR 47: 1049-1054; de Quadros et al., 1998, Measles eradication: experience in the Americas. Bull World Health Organ 76, Suppl 2: 47-52; Gans et al. 2001). New vaccines should be able to be administered to young children within the first 2-3 months. To fulfill this requirement, the vaccine needs to be extremely safe and efficacious. Additionally new vaccines must be able to induce an immune response in individuals with an immature immune system and in the presence of maternal antibodies.

The most promising vector candidates for a new measles vaccine are based on the replication-deficient MVA virus. The safety and immunogenicity of an MVA-based strain as a potential measles vaccine have been evaluated in several animal studies (Stittelaar et al., 2000, Protective Immunity in Macaques Vaccinated with a Modified Vaccinia Virus Ankara-Based Measles Virus Vaccine in the Presence of Passively Acquired Antibodies. J. Virol. 74: 4236-4243; Stittelaar et al., 2001, Safety of modified vaccinia virus Ankara (MVA) in immune-suppressed macaques. Vaccine 19: 3700-9; Weidinger et al., 2001, Vaccination with recombinant modified vaccinia virus Ankara protects against measles virus infection in the mouse and cotton rat model. Vaccine 19: 2764-2768; and Zhu et al., 2000, Evaluation of Recombinant Vaccinia Virus Measles Vaccines in Infant Rhesus Macaques with Preexisting Measles Antibody. Virology 276: 202-213).

Stittelaar et al., 2000, used a recombinant modified vaccinia virus Ankara (MVA), encoding the measles virus (MV) fusion (F) and hemagglutinin (H) (MVA-FH) glycoproteins, in an MV vaccination-challenge model with macaques. Animals were vaccinated twice in the absence or presence of passively transferred MV-neutralizing macaque antibodies and challenged 1 year later intratracheally with wild-type MV. After the second vaccination with MVA-FH, all the animals developed MV-neutralizing antibodies and MV-specific T-cell responses. Although MVA-FH was slightly less effective in inducing MV-neutralizing antibodies in the absence of passively transferred antibodies than the currently used live attenuated vaccine, it proved to be more effective in the presence of such antibodies. All vaccinated animals were effectively protected from the challenge infection.

Weidinger et al., 2001, tested the safety and immunogenicity of a recombinant virus expressing the hemagglutinin of measles virus (MVA-MV-H) using the mouse model of measles virus induced encephalitis and the cotton rat model for respiratory infection, which is very sensitive to infection with replication competent vaccinia virus. MVA-MV-H induced a TH1 response, neutralizing antibodies and conferred protection against both encephalitis and lung infection. In these animals MVA-MV-H proved to be a very well tolerated vaccine. However, the efficiency in the presence of MV specific maternal antibodies was low (even using a prime-boost strategy).

Since immunization of newborn infants with standard measles vaccines is not effective because of the presence of maternal antibody, Zhu et al., 2000, immunized newborn rhesus macaques with recombinant vaccinia viruses expressing measles virus hemagglutinin (H) and fusion (F) proteins, using the replication-competent WR strain of vaccinia virus or the replication defective MVA strain. The infants were boosted at 2 months and then challenged intranasally with measles virus at 5 months of age. Some of the newborn monkeys received measles immune globulin (MIG) prior to the first immunization, and these infants were compared to additional infants that had maternal measles-neutralizing antibody. In the absence of measles antibody, vaccination with either vector induced neutralizing antibody, cytotoxic T cell (CTL) responses to measles virus and protection from systemic measles infection and skin rash. The infants vaccinated with the MVA vector developed lower measles-neutralizing antibody titers than those vaccinated with the WR vector, and they sustained a transient measles viremia upon challenge. Either maternal antibody or passively transferred MIG blocked the humoral response to vaccination with both WR and MVA, and the frequency of positive CTL responses was reduced. Despite this inhibition of vaccine-induced immunity, there was a reduction in peak viral loads and skin rash after measles virus challenge in many of the infants with preexisting measles antibody.

Based on the above, there is a need in the art for measles vaccines offering better protection in children less than 15 months in age and providing increased protection for older children and adults.

### DETAILED DESCRIPTION OF THE INVENTION

According to the present invention, a recombinant Modified Vaccinia virus Ankara, MVA, encoding 3 genes of the measles virus, namely H (hemagglutinin protein), F (fusion protein) and N (nucleoprotein) has been generated. The hemagglutinin protein is a surface glycoprotein responsible for binding of the measles virus to suitable receptors on host cells. The fusion protein is also on the surface of the measles virus and responsible for fusion of the viral envelope with the target cell membrane. H is an essential cofactor for promoting fusion and H and F together are responsible for immunosuppressive properties of the measles virus. The nucleoprotein N belongs to the structural proteins and is responsible for encapsulation of the measles genome.

MVA originates from the dermal vaccinia strain Ankara (Chorioallantois Vaccinia Ankara (CVA) virus) that was maintained in the Vaccination Institute, Ankara, Turkey for many years and used as the basis for vaccination of humans. However, due to the often severe post-vaccinal complications associated with vaccinia viruses, there were several attempts to generate a more attenuated, safer smallpox vaccine.

During the period of 1960 to 1974, Prof. Anton Mayr succeeded in attenuating CVA by over 570 continuous passages in CEF cells (Mayr et al., 1975, Passage History: Abstammung, Eigenschaften and Verwendung des attenuierten Vaccina-Stammes MVA. Infection 3: 6-14). As part of the early development of MVA as a pre-smallpox vaccine, there were clinical trials using MVA-517 (corresponding to the 517^{th} passage) in combination with Lister Elstree (Stickl, 1974, Smallpox vaccination and its consequences: first experiences with the highly attenuated smallpox vaccine “MVA”. Prev. Med. 3(1): 97-101; Stickl and Hochstein-Mintzel, 1971, Intracutaneous smallpox vaccination with a weak pathogenic vaccinia virus (“MVA virus”). Munch Med Wochenschr. 113: 1149-1153) in subjects at risk for adverse reactions from vaccinia. In 1976, MVA derived from MVA-571 seed stock (corresponding to the 571^{st} passage) was registered in Germany as the primer vaccine in a two-stage parenteral smallpox vaccination program. Subsequently, MVA-572 was used in approximately 120,000 Caucasian individuals, the majority children between 1 and 3 years of age, with no reported severe side effects, even though many of the subjects were among the population with high risk of complications associated with vaccinia (Mayr et al., 1978, The smallpox vaccination strain MVA: marker, genetic structure, experience gained with the parenteral vaccination and behaviour in organisms with a debilitated defense mechanism (author's transl). Zentralbl. Bacteriol. (B) 167: 375-390). MVA-572 was deposited at the European Collection of Animal Cell Cultures as ECACC V94012707.

Being that many passages were used to attenuate MVA, there are a number of different strains or isolates, depending on the passage number in CEF cells. All MVA strains originate from Dr. Mayr and most are derived from MVA-572 that was used in Germany during the smallpox eradication program, or MVA-575 that was extensively used as a veterinary vaccine. MVA-575 was deposited on Dec. 7, 2000, at the European Collection of Animal Cell Cultures (ECACC) with the deposition number V00120707. The MVA-BN® product used to generate recombinant MVA according to the present invention (MVA-mBN85B) is derived from MVA-584 (corresponding to the 584^{th} passage of MVA in CEF cells).

By serial propagation (more than 570 passages) of the CVA on primary chicken embryo fibroblasts, the attenuated CVA-virus MVA (Modified Vaccinia Virus Ankara) was obtained. MVA was further passaged by Bavarian Nordic and is designated MVA-BN®, corresponding to passage 583. MVA as well as MVA-BN® lacks approximately 15% (31 kb from six regions) of the genome compared with ancestral CVA virus (FIG. 1). The deletions affect a number of virulence and host range genes, as well as the gene for Type A inclusion bodies. A sample of MVA-BN® was deposited on Aug. 30, 2000 at the European Collection of Cell Cultures (ECACC) under number V00083008.

MVA-BN® can attach to and enter human cells where virally-encoded genes are expressed very efficiently. However, assembly and release of progeny virus does not occur. Preparations of MVA-BN® and derivatives have been administered to many types of animals, and to more than 2000 human subjects, including immunodeficient individuals. All vaccinations have proven to be generally safe and well tolerated.

The perception from many different publications is that all MVA strains are the same and represent a highly attenuated, safe, live viral vector. However, preclinical tests have revealed that MVA-BN® demonstrates superior attenuation and efficacy compared to other MVA strains (WO 02/42480): The MVA variant strains MVA-BN® as, e.g., deposited at ECACC under number V00083008 have the capability of reproductive replication in vitro in chicken embryo fibroblasts (CEF), but no capability of reproductive replication in the human keratinocyte cell line HaCat, the human embryo kidney cell line 293, the human bone osteosarcoma cell line 143B, and the human cervix adenocarcinoma cell line HeLa. Further, MVA-BN® strains fail to replicate in a mouse model that is incapable of producing mature B and T cells, and as such is severely immune compromised and highly susceptible to a replicating virus. An additional or alternative property of MVA-BN® strains is the ability to induce at least substantially the same level of immunity in vaccinia virus prime/vaccinia virus boost regimes when compared to DNA-prime/vaccinia virus boost regimes.

The term “not capable of reproductive replication” is used in the present application as defined in WO 02/42480 and U.S. Pat. No. 6,761,893, respectively. Thus, said term applies to a virus that has a virus amplification ratio at 4 days after infection of less than 1 using the assays described in U.S. Pat. No. 6,761,893, which assays are hereby incorporated by reference. The “amplification ratio” of a virus is the ratio of virus produced from an infected cell (Output) to the amount originally used to infect the cells in the first place (Input). A ratio of “1” between Output and Input defines an amplification status wherein the amount of virus produced from the infected cells is the same as the amount initially used to infect the cells.

MVA-BN® or its derivatives are, according to one embodiment, characterized by inducing at least substantially the same level of immunity in vaccinia virus prime/vaccinia virus boost regimes when compared to DNA-prime/vaccinia virus boost regimes. A vaccinia virus is regarded as inducing at least substantially the same level of immunity in vaccinia virus prime/vaccinia virus boost regimes when compared to DNA-prime/vaccinia virus boost regimes if the CTL response as measured in one of the “assay 1” and “assay 2” as disclosed in WO 02/42480, preferably in both assays, is at least substantially the same in vaccinia virus prime/vaccinia virus boost regimes when compared to DNA-prime/vaccinia virus boost regimes. More preferably the CTL response after vaccinia virus prime/vaccinia virus boost administration is higher in at least one of the assays, when compared to DNA-prime/vaccinia virus boost regimes. Most preferably the CTL response is higher in both assays.

WO 02/42480 discloses how Vaccinia viruses are obtained having the properties of MVA-BN®. The highly attenuated MVA-BN virus can be derived, e.g., by the further passage of a Modified Vaccinia virus Ankara (MVA), such as MVA-572 or MVA-575.

In summary, MVA-BN® has been shown to have the highest attenuation profile compared to other MVA strains and is safe even in severely immunocompromised animals.

Although MVA exhibits strongly attenuated replication in mammalian cells, its genes are efficiently transcribed, with the block in viral replication being at the level of virus assembly and egress. (Sutter and Moss, 1992, Nonreplicating vaccinia vector efficiently expresses recombinant genes. Proc. Natl. Acad. Sci. U.S.A 89: 10847-10851; Carroll and Moss, 1997, Host range and cytopathogenicity of the highly attenuated MVA strain of vaccinia virus: propagation and generation of recombinant viruses in a nonhuman mammalian cell line. Virology 238: 198-211.) Despite its high attenuation and reduced virulence, in preclinical studies MVA has been shown to elicit both humoral and cellular immune responses to vaccinia and genes cloned into the MVA genome (Harrer et al., 2005, Therapeutic Vaccination of HIV-1-infected patients on HAART with recombinant HIV-1 nef-expressing MVA: safety, immunogenicity and influence on viral load during treatment interruption. Antiviral Therapy 10: 285-300; Cosma et al., 2003, Therapeutic vaccination with MVA-HIV-1 nef elicits Nef-specific T-helper cell responses in chronically HIV-1 infected individuals. Vaccine 22(1): 21-29; Di Nicola et al., 2003, Clinical protocol. Immunization of patients with malignant melanoma with autologous CD34(+) cell-derived dendritic cells transduced ex vivo with a recombinant replication-deficient vaccinia vector encoding the human tyrosinase gene: a phase I trial. Hum Gene Ther. 14(14): 1347-1360; Di Nicola et al., 2004, Boosting T cell-mediated immunity to tyrosinase by vaccinia virus-transduced, CD34(+)-derived dendritic cell vaccination: a phase I trial in metastatic melanoma. Clin Cancer Res. 10(16): 5381-5390).

MVA and recombinant MVA-based vaccines can be generated, passaged, produced and manufactured in CEF cells cultured in serum-free medium. Many recombinant MVA-BN® variants have been characterized for preclinical and clinical development. No differences in terms of the attenuation (lack of replication in human cell lines) or safety (preclinical toxicity or clinical studies) have been observed between MVA-BN®, the viral vector backbone, and the various recombinant MVA-based vaccines.

The safety and immunogenicity of MVA-BN® and recombinant MVA-BN® vaccines have been demonstrated in more than 15 completed or on-going clinical trials in healthy subjects, people diagnosed with atopic dermatitis, HIV infected people and cancer (melanoma) patients. Thus, MVA-BN® is used as a preferred vector for generating the recombinant virus.

However, also vaccines based on other MVA viral strains as, for example, MVA-572 or -575, are suitable for generating a recombinant MVA including the 3 above-mentioned measles virus antigens according to the present invention. Unexpectedly, when all three antigens are included in the MVA viral vector improved protection is found: Preclinical studies performed to date have shown that the recombinant MVA is safe, non-toxic, well tolerated, and immunogenic in all age groups tested as summarized in the following points:
Repeat administrations of the recombinant MVA to investigate toxicity and local tolerance of the vaccine demonstrated the vaccine to be safe and well tolerated in adult and juvenile rats. All of the observed side effects of the recombinant MVA were considered minimal and were demonstrated to be reversible following the last vaccination.
The recombinant MVA vaccine was shown to induce antibody responses to the measles virus in both juvenile and adult rats.
Recombinant MVA including all three antigens induced even superior cellular and humoral responses to the measles virus in adult mice when compared to Measles vaccine Merieux® (Rouvax, Schwartz strain 1000 TCID₅₀).
Both T cell and B cell immune responses to the recombinant MVA were observed not only in adult animals, but also in newborn and juvenile mice. Only a single immunization with the recombinant viral vector was required to induce the B cell immune response. The B cell immune response in newborns after a single immunization was stable for more than 189 days after immunization.
Preliminary results of the first Phase I clinical trial in adult humans (18-32 years old) showed that the MVA vaccine induces a strong immune response and is safe and well tolerated.

BALB/c mice are able to mount a low, but detectable, measles-specific IgG response after two s.c. administrations of 10⁶ TCID₅₀ of the recombinant virus. Application of a ten-fold higher dose of the recombinant MVA resulted in approximately 1000-fold higher Measles-specific mean IgG responses and antibody titers were already detected after the first administration of the vaccine. Another ten-fold increase in the vaccine dose resulted in approximately five times higher specific mean antibody titers.

Thus, these data may indicate that the Measles-specific IgG response reaches saturation when applying doses of the recombinant MVA as high as 10⁸ TCID₅₀. Furthermore, at this high dose of the recombinant a boost of the humoral immune response was primarily detected following the second administration, whereas lower boost effects were determined after the third and the fourth administration.

Compared to the doses of 10⁷ or 10⁸ TCID₅₀ of MVA-Measles, the humoral immune response induced by the commercially available vaccine (Rouvax; Schwartz strain 1000 TCID₅₀) was substantially lower. The lower humoral immune response of Rouvax is surprising since the commercial vaccine consists of the whole virus. This difference cannot appropriately be explained by differences in the identity of the differently used virus strains: Comparing the Schwartz strain with the Khartoum SUD/34.97 strain revealed a homology of 97%, 97%, and 98% for the nucleocapsid, the hemagglutinin, and for the fusion protein, respectively. In addition to the lower immune response, the sero-conversion rate was substantially lower in the group administered with the Measles vaccine Merieux® (40%) compared to the one detected with the two highest doses of MVA-Measles (100%) thereby demonstrating the superior quality of MVA-Measles.

N-protein specific cellular immune responses were detected in the recombinant MVA-Measles vaccinated mice. It is surprising to find the highest mean values of IFNγ secreting cells not in the group administered with 10⁸ TCID₅₀, but in the one administered with 10⁷ TCID₅₀. This is in contrast to the dose-dependency detected by the Measles specific IgG response.

The absence of a cellular immune response following administration of the Measles vaccine Merieux® may be due to two reasons: First, this vaccine group was included into the study to obtain humoral responses and the vaccination schedule was therefore applied to allow an 11-week interval between administration and analysis of the cellular immune response. Second, the commercially available vaccine is based on a Measles virus of the Schwartz strain (Rouvax; Schwartz strain 1000 TCID₅₀) which might slightly differ in the amino acid sequence to strain Khartoum SUD/34.97 which was used to develop the Measles-specific inserts when generating the recombinant MVA.

In summary, it has been found that MVA including the H, F and N gene of measles virus is able to induce Measles-specific humoral immune responses as well as N-protein specific cellular immune responses. In addition, the recombinant virus vaccine is superior to Measles vaccine Merieux® since the conversion rate of the humoral immune response was higher and detected earlier.

Additionally, since MVA-BN® was used as vector for generating the recombinant virus, resulting in MVA-mBN85B, the same excellent attenuation profile as the viral vector MVA-BN® was found: Also MVA-mBN85B has shown an inability to cause cell fusion. The recombinant also failed to reproductively replicate in human cells. In human cells the viral genes are expressed, but no infectious virus is produced. The restricted host range of MVA-BN® may explain the non-virulent phenotype observed in vivo in a wide range of mammalian species including humans. Some key features of MVA-BN® that make this a promising vaccine vector include:
As already mentioned above, MVA-BN® fails to reproductively replicate in human cell lines or mammalians, even in severely immune suppressed mice.
MVA-BN® has been shown to be safe in numerous toxicity studies, including repeated toxicity exposure in rabbits as well as peri- and post-natal teratology studies in pregnant dams and pups, and MVA-BN® has been shown to be rapidly cleared (within 48 hours post vaccination) from rabbits in a biodistribution study.
MVA-BN® can be used in homologous prime-boost regimes even in the presence of a pre-existing immunity to the viral vector.
More than 2000 people have been safely vaccinated with MVA-BN® or recombinant MVA-based vaccines, including healthy subjects, Human Immunodeficiency Virus (HIV) infected people (CD4 cells >350/μl) and people diagnosed with Atopic Dermatitis (AD).

The recombinant MVA-mBN85B reveals the same properties as MVA-BN® strains and the deposited strain V0083008, respectively. In particular,
the recombinant virus fails to reproductively replicate in vitro in human cell lines.
the recombinant virus fails to reproductively replicate in vivo in humans and mice, even in severely immune suppressed mice.
the recombinant virus has a virus amplification ratio at least two fold less than MVA-575 in HeLa and HaCaT cell lines.
the recombinant virus has the capacity to reproductively replicate in chicken embryo fibroblast cells.

Thus, the recombinant MVA including the measles virus antigens H, F and N is a Highly Attenuated Modified Vaccinia virus Ankara (“HA-MVA”). HA-MVA viruses reveal the same characteristics as mentioned above for the recombinant MVA mBN85B virus, namely:
An HA-MVA virus fails to reproductively replicate in vitro in human cell lines.
An HA-MVA virus fails to reproductively replicate in vivo in humans and mice, even in severely immune suppressed mice.
An HA-MVA virus has a virus amplification ratio at least two fold less than MVA-575 in Hela and HaCaT cell lines.
An HA-MVA virus has the capacity to reproductively replicate in chicken embryo fibroblast cells.

The term “fails to reproductively replicate” applies to a virus that has a virus amplification ratio at 4 days after infection of less than 1 using the assays described in U.S. Pat. No. 6,761,893, which assays are hereby incorporated by reference. The “amplification ratio” of a virus is the ratio of virus produced from an infected cell (Output) to the amount originally used to infect the cells in the first place (Input). A ratio of “1” between Output and Input defines an amplification status wherein the amount of virus produced from the infected cells is the same as the amount initially used to infect the cells.

HA-MVA viruses include MVA-BN and recombinant viruses derived from MVA-BN, for example, by insertion of a heterologous gene under the control of, preferably, a poxvirus promoter. The recombinant MVA-BN virus according to the present invention is a derivative of MVA-BN®. “Derivatives” of MVA-BN® refer to viruses exhibiting essentially the same replication characteristics as MVA-BN®, but exhibiting differences in one or more parts of their genomes.

Preferably, the recombinant MVA according to the present invention has a virus amplification ratio at least three fold less than MVA-575 in HeLa and HaCaT cell lines and, as a further embodiment, has an amplification ratio of greater than 500 in CEF cells.

However, as already stated above, also vaccines based on other MVA viral strains, like MVA-572 or -575, can be used as viral vector backbone for generating the recombinant vaccine strain.

The recombinant MVA virus according to the present invention can be generated by routine methods known in the art. For example, the MVA virus can be generated by following the procedures set out in the Examples.

Methods to obtain recombinant poxviruses or to insert exogenous coding sequences into a poxviral genome are well known to the person skilled in the art. For example, methods are described in the following references: Molecular Cloning, A laboratory Manual. Second Edition. By J. Sambrook, E. F. Fritsch and T. Maniatis. Cold Spring Harbor Laboratory Press. 1989: describes techniques for standard molecular biology techniques such as cloning of DNA, DNA and RNA isolation, western blot analysis, RT-PCR and PCR amplification techniques. Virology Methods Manual. Edited by Brian W J Mahy and Hillar O Kangro. Academic Press. 1996: describes techniques for the handling and manipulation of viruses. Molecular Virology: A Practical Approach. Edited by A J Davison and R M Elliott. The Practical Approach Series. IRL Press at Oxford University Press. Oxford 1993. Chapter 9: Expression of genes by Vaccinia virus vectors. Current Protocols in Molecular Biology. Publisher: John Wiley and Son Inc. 1998. Chapter 16, section IV: Expression of proteins in mammalian cells using Vaccinia viral vector: describes techniques and know-how for the handling, manipulation and genetic engineering of MVA.

For the generation of recombinant poxviruses according to the present invention, different methods may be applicable. The DNA sequence to be inserted into the virus can be placed into an E. coli plasmid construct into which DNA homologous to a section of DNA of the poxvirus has been inserted. Separately, the DNA sequence to be inserted can be ligated to a promoter. The promoter-gene linkage can be positioned in the plasmid construct so that the promoter-gene linkage is flanked on both ends by DNA homologous to a DNA sequence flanking a region of pox DNA containing a non-essential locus. The resulting plasmid construct can be amplified by growth within E. coli bacteria and isolated. The isolated plasmid containing the DNA gene sequence to be inserted can be transfected into a cell culture, e.g., chicken embryo fibroblasts (CEFs), along with the poxvirus. Recombination between homologous pox DNA in the plasmid and the viral genome, respectively, can generate a poxvirus modified by the presence of foreign DNA sequences.

According to a preferred embodiment, a cell of a suitable cell culture as, e.g., CEF cells, can be infected with a poxvirus. The infected cell can be, subsequently, transfected with a first plasmid vector comprising the foreign gene, preferably under the transcriptional control of a poxvirus expression control element. As explained above, the plasmid vector also comprises sequences capable of directing the insertion of the exogenous sequence into a selected part of the poxviral genome. Optionally, the plasmid vector also contains a cassette comprising a marker and/or selection gene operably linked to a poxviral promoter. Suitable marker or selection genes are, e.g., the genes encoding the Green Fluorescent Protein, β-Galactosidase, neomycin, phosphoribosyltransferase or other markers. The use of selection or marker cassettes simplifies the identification and isolation of the generated recombinant poxvirus. However, a recombinant poxvirus can also be identified by PCR technology.

In one embodiment, a single DNA molecule comprises the nucleic acid encoding hemagglutinin protein (H), fusion protein (F), and nucleoprotein (N) of the measles virus. In an other embodiment, two or three different DNA molecules comprise the nucleic acid encoding hemagglutinin protein (H), fusion protein (F), and nucleoprotein (N) of the measles virus. In a further embodiment, the recombinant MVA-Measles virus comprises SEQ ID NO:1, SEQ ID NO:2, and SEQ ID NO:3.

The hemagglutinin protein (H), fusion protein (F), and nucleoprotein (N) of the measles virus can be derived from a measles virus strain, for example, using RT PCR techniques. In preferred embodiments, the measles virus strain is a WTF, TYCSA, CAM-70, Edmonston, L-16, Sugiyama, AIK-C, Toyoshima, Mantooth, Halle, Schwartz, or Khartoum SUD/34.97 strain.

In a further embodiment, expression of the H (hemagglutinin protein), F (fusion protein), or N (nucleoprotein) of the measles virus is under the control of one or more poxvirus promoters. In a preferred embodiment, the poxvirus promoter is a cowpox virus ATI promoter. In a particularly preferred embodiment, expression of the H (hemagglutinin protein), F (fusion protein), and N (nucleoprotein) of the measles virus is under the control of cowpox virus ATI promoters. In one embodiment, the promoter comprises SEQ ID NO:4.

The genes encoding H (hemagglutinin protein), F (fusion protein), and N (nucleoprotein) of the measles virus may be inserted into a non-essential region of the virus genome as, for example, at a naturally occurring deletion site of the MVA genome (disclosed in WO 97/02355). Preferably, the heterologous nucleic acid sequences are inserted into an intergenic region of the MVA genome (disclosed in WO 03/097845). In a further preferred embodiment, the antigens of the measles virus are inserted into intergenic regions IGR 64/65, IGR07/08, and IGR 44/45 of the genome.

As an alternative or in addition to the H, F, and N antigens one or more antigenic epitopes of one, two or all of the measles virus antigens are inserted into the viral genome. “Epitopes”, also known as antigenic determinants, are part of an antigen and shorter stretches that still elicit an immune response. Epitopes can be mapped using protein microarrays, and with the ELISPOT or ELISA technique. Epitope mapping is, thus, the process of identification and characterization of the minimum molecular structures that are able to be recognized by the immune system elements, mainly T and B cells. A collection of in vivo and in vitro methodologies are used for epitope mapping and are well known to the skilled practitioner. Among the most used are binding assay, ELISPOT, HLA transgenic mice and prediction software. Additionally, databases for T and B cell epitopes are already available.

Preferably, the recombinant MVA virus according to the present invention does not induce cell fusion in human cell lines. Preferably, the recombinant virus does not induce cell fusion in HeLa or HUVEC cells.

As already mentioned above, although MVA-mBN85B was generated by cloning Measles virus genes into MVA-BN®, other MVA viruses can be used for the expression of Measles virus genes. These other MVA viruses can be generated by many routine techniques known in the art. Other MVA strains, such as MVA-575 or MVA-572, may also be attenuated and, thus, will subsequently reveal the same properties as the highly attenuated MVA-BN® strain. For this, the MVA strains are cultured in permissive cells, and viruses are selected by assessing attenuation, such as growth on human cell lines, e.g., HeLa and HaCaT.

The growth of MVA in culture can lead to mutations in the genome of the MVA. By using the appropriate selection procedures (i.e., growth on particular cell lines), the desired phenotype can be maintained, while allowing mutations that do not affect these properties. Methods for growing MVA on various cell lines are well known in the art and are exemplified in the Examples.

The additions of mutagens to the media in which the viruses are grown can facilitate the generation of mutations in the genome of an MVA virus. Similarly, PCR and other molecular techniques can be used to introduce mutations into the genome of the MVA. These mutations can be targeted to non-essential regions of the genome or can be randomly generated.

Since the virus used as a vector according to the invention is—dependent on the strain used—more or less growth restricted and, thus, attenuated, it is an ideal candidate for the treatment of a wide range of mammals including humans and even immune-compromised humans. Hence, the present invention also provides a pharmaceutical composition and also a vaccine for inducing an immune response in a living animal body, including a human.

The vaccine preferably comprises the recombinant MVA viruses in a concentration range of 10⁴ to 10⁹ TCID (tissue culture infectious dose)₅₀/ml, preferably in a concentration range of 10⁵ to 5×10⁸ TCID₅₀/ml, more preferably in a concentration range of 10⁶ to 10⁸ TCID₅₀/ml, and most preferably in a concentration range of 10⁷ to 10⁸ TCID₅₀/ml.

A preferred vaccination dose for humans comprises 10⁶ to 10⁹ TCID₅₀, most preferably a dose of 10⁷ TCID₅₀ or 10⁸ TCID₅₀.

The pharmaceutical composition may generally include one or more pharmaceutically acceptable and/or approved carriers, additives, antibiotics, preservatives, adjuvants, diluents and/or stabilizers. Such auxiliary substances can be water, saline, glycerol, ethanol, wetting or emulsifying agents, pH buffering substances, or the like. Suitable carriers are typically large, slowly metabolized molecules such as proteins, polysaccharides, polylactic acids, polyglycolic acids, polymeric amino acids, amino acid copolymers, lipid aggregates, or the like.

For the preparation of vaccines, the recombinant MVA virus according to the invention can be converted into a physiologically acceptable form. This can be done based on the experience in the preparation of poxvirus vaccines used for vaccination against smallpox (as described by Stickl et al. 1974).

For example, the purified virus can be stored at −80° C. with a titre of 5×10⁸ TCID₅₀/ml formulated in about 10 mM Tris, 140 mM NaCl pH 7.4. For the preparation of vaccine shots, e.g., 10²-10⁸ particles of the virus can be lyophilized in 100 ml of phosphate-buffered saline (PBS) in the presence of 2% peptone and 1% human albumin in an ampoule, preferably a glass ampoule. Alternatively, the vaccine shots can be produced by stepwise freeze-drying of the virus in a formulation. This formulation can contain additional additives such as mannitol, dextran, sugar, glycine, lactose or polyvinylpyrrolidone or other aids such as antioxidants or inert gas, stabilizers or recombinant proteins (e.g. human serum albumin) suitable for in vivo administration. The glass ampoule is then sealed and can be stored between 4° C. and room temperature for several months. However, as long as no need exists the ampoule is stored preferably at temperatures below −20° C.

For vaccination or therapy, the lyophilisate can be dissolved in an aqueous solution, preferably physiological saline or Tris buffer, and administered either systemically or locally, i.e. parenteral, subcutaneous, intravenous, intramuscular, or any other path of administration know to the skilled practitioner. The mode of administration, the dose and the number of administrations can be optimized by those skilled in the art in a known manner. However, most commonly a patient is vaccinated with a second shot about one month to six weeks after the first vaccination shot.

The invention further provides kits comprising the recombinant MVA virus according to the present invention. The kit can comprise one or multiple containers or vials of the recombinant MVA virus, together with instructions for the administration of the virus to a subject. In a preferred embodiment, the subject is a human. The instructions can indicate that the recombinant MVA virus is administered to the subject in a single dosage, or in multiple (i.e., 2, 3, 4, etc.) dosages. The instructions can indicate that the MVA virus is administered in a first (priming) and second (boosting) administration. The kit comprises, in a further embodiment, the recombinant MVA (or the pharmaceutical composition or vaccine comprising the recombinant MVA) for a first inoculation (“priming inoculation”) in a first vial/container and for a second inoculation (“boosting inoculation”) in a second vial/container.

The invention provides methods for immunizing an animal body, including a human. In one embodiment a subject mammal, which includes rats, rabbits, mice, and humans are immunized comprising administering a dosage of a recombinant MVA to the subject, preferably to a human. In one embodiment, the subject is an adult. In other embodiments, the subject's age can be less than 15 months, less than 12 months, less than 9 months, less than 6, or less than 3 months. In other embodiments, the subject's age can be from 0-3 months, 3-6 months, 6-9 months, 9-12 months, or 12-15 months.

Preferably, a dosage of the recombinant MVA-Measles virus of 10⁶ to 10⁹ TCID₅₀ is administered to the subject. More preferably, a dosage of 10⁶ to 5×10⁸ TCID₅₀ is administered to the subject. Most preferably, a dosage of 10⁷ to 10⁸ TCID₅₀ is administered to the subject. A preferred dosage for humans comprises 10⁷ TCID₅₀ or 10⁸ TCID₅₀ of the recombinant MVA virus.

The MVA virus according to the present invention can be administered to the subject in a single dosage, or in multiple (i.e., 2, 3, 4, etc.) dosages. The MVA virus can be administered in a first (priming) and second (boosting) administration. In one embodiment, the first dosage comprises 10⁷ to 10⁸ TCID₅₀ of the recombinant MVA virus and the second dosage comprises 10⁷ to 10⁸ TCID₅₀ of the virus.

The immunization can be administered either systemically or locally, i.e. parenteral, subcutaneous, intravenous, intramuscular, or any other path of administration known to the skilled practitioner.

In one embodiment, a single immunization with the recombinant MVA virus according to the present invention induces a Measles ELISA geometric mean titer (GMT) at least 10-fold greater than that induced by a single immunization with Rouvax vaccine of Merieux® (Schwartz strain 1000 TCID₅₀) in mice.

In a further embodiment, a single immunization with the recombinant MVA virus induces a Measles ELISA geometric mean titer (GMT) at least 2-fold greater than that induced by a single immunization with Rouvax vaccine of Merieux® (Schwartz strain 1000 TCID₅₀) in humans.

In one embodiment, the invention encompasses a method of generating a recombinant virus encoding the hemagglutinin protein (H), fusion protein (F), and nucleoprotein (N) of the measles virus and/or encoding an antigenic epitope of one or more of said measles virus antigens, said method comprising inserting the H, F, and N genes and/or the antigenic epitope(s) into the MVA viral genome.

In particular, the method comprises the steps of:
a) inserting nucleic acid encoding hemagglutinin protein (H), fusion protein (F), and nucleoprotein (N) of the measles virus and/or an antigenic epitope of one, two or all three of said measles virus antigens into the MVA strain; and
b) determining that a single immunization with the recombinant MVA virus induces a Measles ELISA geometric mean titer (GMT) which is 10-fold greater than that induced by a single immunization with Rouvax vaccine of Merieux® (Schwartz strain 1000 TCID₅₀) in mice and/or
c) determining that a single immunization with the recombinant MVA virus induces a Measles ELISA geometric mean titer (GMT) which is 2-fold greater than that induced by a single immunization with Rouvax vaccine of Merieux® (Schwartz strain 1000 TCID₅₀) in humans.

The present invention also encompasses a method of producing the H, F, and/or N antigen of a measles virus and/or an antigenic epitope of one, two, or all three of said measles virus antigens and/or the recombinant virus according to the present invention, said method comprising
a) infecting a cell with the recombinant virus;
b) cultivating the infected cell under suitable conditions; and
c) isolating and/or enriching the antigen and/or the antigenic epitope(s) and/or the virus produced by said cell.

Further encompassed is a method of introducing an H, F, and N antigen of a measles virus and/or an antigenic epitope of one or more or all of said measles virus antigens into a cell comprising infecting the cell with the recombinant MVA virus according to the present invention.

The present invention also relates to a cell comprising the recombinant MVA according to the invention and as described, respectively.

### BRIEF DESCRIPTION OF THE DRAWINGS

The invention may be more fully understood with reference to the drawings, in which:

FIG. 1 depicts the localization of the six deletion sites in the MVA genome compared to CVA. Letters A to P identify HindIII restriction enzyme digestion fragments. The positions of the CVA sequences that are lacking in the MVA genome (deletions I to VI) are shown.

FIG. 2 depicts a schematic map of MVA genome (HindIII restriction map indicated by letters A to P outlining the IGR 64/65, IGR 07/08 and IGR 44/45 sites used for generation of MVA-mBN85B).

FIGS. 3A-C depict maps of the recombination plasmids pBNX87, pBNX86, and pBNX118. MVA-BN® DNA sequences adjacent to IGR 64/65 (pBNX118), 44/45 (pBNX87) and 07/08 (pBNX86) were cloned to allow recombination into the MVA-BN® genome. An expression cassette for NPTII/EGFP (pBNX87, pBNX86) or Ecogpt/RFP (pBNX118) under the control of the well characterized strong synthetic Vaccinia virus promoter (Ps) was inserted between the MVA-BN® DNA flanking sequences. Further, an IRES element was added in front of the EGFP gene to generate a bicistronic cassette to allow expression of NPTII and EGFP from a single promoter whereas an additional Ps promoter was inserted in front of the RFP gene.

FIG. 4 depicts final recombination plasmid pBN133 containing the F gene. The F gene under the control of the ATI promoter (pr ATI) was inserted into a PacI site in pBNX86 to generate the final recombination plasmid pBN133.

FIG. 5 depicts final recombination plasmid pBN135 containing the H gene. The H gene under the control of the ATI promoter (prATI) was inserted into a PacI site in pBNX118 to generate the final recombination plasmid pBN135.

FIG. 6 depicts final recombination plasmid pBN157 containing the N gene. The N gene under the control of the ATI promoter (prATI) was inserted into a PacI site in pBNX87 to generate the final recombination plasmid pBN157.

FIG. 7 depicts a Flow Chart of the Process Followed to Generate MVA-mBN85B.

FIG. 8 depicts F, H and N Specific Insert PCR. The presence of the F, H and N gene was confirmed by an insert specific PCR. Material from PreMaster DNA was extracted from MVA-mBN85B (250 μl), eluted in 50 μl, and 1 μl was analysed by PCR. For the plasmid control, 10 ng plasmid DNA and for MVA-BN® (BN), DNA from the equivalent of 1×10⁴ TCID₅₀ were analysed. Test sample MVA-mBN85B PreMaster PP5 was used for production. PreMasters of PP4 and PP6 were back-ups.

FIG. 9 depicts MVA-mBN68, MVA-mBN75A and MVA-mBN85B IGR 07/08, IGR 64/65 and IGR 44/45 specific PCR. The elimination of MVA-BN empty plasmid virus and correct insertion of the F, H and N gene into IGR 07/08, IGR 64/65 and IGR 44/45 was confirmed by PCR. DNA was prepared from MVA-mBN68, MVA-mBN75A and MVA-mBN85A (250 μl), eluted in 50 μl, and 1 μl was analysed by PCR. For MVA-BN® (BN), DNA from the equivalent of 1×10E⁴ TCID₅₀ was analyzed, and 10 ng of pBN146 plasmid DNA was analysed.

FIG. 10 depicts nested PCR for NPTII/EGFP and gpt/RFP for MVA-mBN85B. A nested PCR was performed. DNA extracted from 250 μl of MVA-mBN85B was eluted into 50 μl, and 0.2 μl or 2 μl was analysed by PCR. MVA-mBN85A was used as a positive control, and water served as negative control. In the positive control, the expected 888 bp (first PCR) and 638 bp (second PCR) bands were clearly visible. As expected, no band was seen with the test sample MVA-mBN85B. Clone # 3, 4, 5 and 6 represent the different PreMasters generated. MVA-mBN85B clone# 6 was chosen for MVB production. +=MVA-mBN85A, positive control.

FIG. 11 is a schematic map of the MVA-BN® genome (HindIII restriction map, indicated by letters A to P) outlining the recombinant inserts cloned in the Intergenic regions 07/08 (F gene), 64/65 (H gene), and 44/45 (N gene) with each under the control of the cowpox virus ATI promoter.

FIG. 12 depicts attenuation profile of MVA-mBN85B. CEF cells and the human cell lines 143B, HaCaT, HeLa, 293 and MRC-5 were infected with MVA-BN and MVA-mBN85B. The amount of virus particles present was determined by a standard titration assay and expressed as the ratio of virus recovered (Day 4) compared to the initial inoculum (Day 0). A ratio of ≦1 is defined as negative for replication.

FIG. 13 depicts humoral immune response in adult rats vaccinated with MVA-mBN85B. Adult Sprague-Dawley rats (n=20 per group at Day −1, n=10 per group at Day 57) were vaccinated s.c. on Days 1 and 29 with either MVA-mBN85B (1×10⁸ TCID₅₀) or TBS as control. Sera were prepared on Day −1 and Day 57. Measles specific IgG immune responses were monitored using an ELISA assay and were expressed as average mIU/ml (milli-International Units of anti-measles IgG were calculated using a human sera standard). Error bars depict the standard error of the mean (SEM).

FIG. 14 depicts humoral immune response in adult mice vaccinated with MVA-mBN85B versus MVA-BN®. Adult BALB/c mice (n=5 per group) were vaccinated s.c. on Days 0, 21, 42 and 63 with either 1×10⁶, 1×10⁷, or 1×10⁸ TCID₅₀ MVA-mBN85B, or TBS or 1×10⁸ TCID₅₀ MVA-BN as control. Sera were prepared on Days −1, 20, 41, 62, and 77 and measles-specific IgG immune responses were monitored using an ELISA assay. The GMT were expressed as mIU/ml (milli-International Units of anti-measles IgG were calculated using a human sera standard), error bars showing ±SEM.

FIG. 15 depicts N-specific cellular immune response in adult mice vaccinated with MVA-mBN85B. Adult BALB/c mice (n=5 per group, except for four mice immunized with 1×10⁶ TCID₅₀) were vaccinated twice s.c. on Days 0 and 21 with either 1×10⁶, 1×10⁷, or 1×10⁸ TCID₅₀ MVA-mBN85B or TBS as control. Splenocytes were prepared on Day 35 and stimulated with two N-specific peptides (peptide 1: YPALGLHEF (SEQ ID NO:5) and peptide 2: YAMGVGVELEN (SEQ ID NO:6). N-specific T cell immune responses were monitored using an IFN-γ ELISpot assay. Average Spot Forming Cells/10⁶ splenocytes ±SEM are shown.

FIG. 16 depicts humoral immune response in adult mice vaccinated with MVA-mBN85B or Measles Vaccine Merieux® (Rouvax®). Adult BALB/c mice (n=5 per group) were vaccinated once (Day 21) or twice (Days 0 and 21) s.c. with 1×10⁸ TCID₅₀ MVA-mBN85B, once with measles vaccine Merieux® on Days 0 or 21 or with TBS as a control. Sera were prepared on Days −1, 14, 20, 28, and 35. Measles specific IgG immune responses were monitored using an ELISA assay. The GMTs were expressed in mIU/ml (milli-International Units of anti-measles IgG were calculated using a human sera standard). Error bars showing ±SEM.

FIG. 17 depicts N-specific cellular immune response in adult mice vaccinated with MVA-mBN85B or Measles Vaccine Merieux®. Adult BALB/c mice (n=5 per group) were vaccinated once (Day 21) or twice (Days 0 and 21) s.c. with 1×10⁸ TCID₅₀ MVA-mBN85B, once with measles vaccine Merieux® on Day 0 or 21 or TBS as control. Splenocytes were prepared on Day 35 and stimulated with two N-specific peptides (peptide 1: YPALGLHEF (SEQ ID NO:5) and peptide 2: YAMGVGVELEN (SEQ ID NO:6)). N-specific T cell immune responses were monitored using an IFN-γ ELISpot assay. Average Spot Forming Cells/10⁶ splenocytes ±SEM are shown.

FIG. 18 depicts humoral immune response in juvenile rats vaccinated with MVA-mBN85B. Juvenile Sprague-Dawley rats (n=20 per group at Day 34, n=10 at Day 62) were vaccinated s.c. on post-natal days (PNDs) 21, 28, and 35 with either MVA-mBN85B (at 1×10⁷ TCID₅₀ or 1×10⁸ TCID₅₀) or TBS as control. Sera were prepared on PNDs 34 and 62. Measles-specific IgG immune responses were monitored using an ELISA assay and expressed as average mIU/ml (milli-International Units of anti-measles IgG were calculated using a human sera standard). Error bars show ±SEM.

FIG. 19 depicts humoral immune response in 7 day old mice vaccinated with MVA-mBN85B or MVA-BN®. 7 days old BALB/c mice (n=5 to 7 per group) were vaccinated once (day 7* or 21*) or twice (day 7* and 21*) s.c. with 1×10⁷ or 1×10⁸ TCID₅₀ MVA-mBN85B, or twice with TBS and 1×10⁸ TCID₅₀ MVA-BN® as control. Half of the mice were sacrificed on Day 35* for analysis of the T cell response. Sera were prepared on Days 20*, 35*, 49*, and 63*. Measles-specific IgG immune responses were monitored using an ELISA assay. The GMTs were expressed as mIU/ml (milli-International Units of anti-measles IgG were calculated using a human sera standard). Error bars showing ±SEM.

*Relative to the day of birth.

FIG. 20 depicts N-specific cellular immune response in 7 day old mice vaccinated with MVA-mBN85B or MVA-BN®. 7 days old BALB/c mice (n=3 to 4 per group) were vaccinated once (Day 7* or 21*) or twice (Day 7* and 21*) s.c. with 1×10⁷ or 1×10⁸ TCID₅₀ MVA-mBN85B or twice with TBS and 1×10⁸ TCID₅₀ MVA-BN® as control. Splenocytes were prepared on Day 35* and stimulated with two N-specific peptides (peptide 1: YPALGLHEF (SEQ ID NO:5) and peptide 2: YAMGVGVELEN(SEQ ID NO:6)). N-specific T cell immune responses were monitored using an IFN-γ ELISpot assay. Average Spot Forming Cells/10⁶ splenocytes ±SEM are shown. They were calculated by subtracting the counts from non-stimulated wells (medium only) from the stimulated ones.

*Relative to the day of birth.

FIG. 21 depicts Humoral Immune Response in Newborn or 7 Days Old Mice Vaccinated with MVA-mBN85B. Newborn or 7 days old BALB/c mice (n=12 per group until Day 35, then n=6) were vaccinated respectively on Day 0* or 7* s.c. with 1×10⁸ TCID₅₀ MVA-mBN85B and then boosted on Day 21*. The control mice received TBS on Days 7* and 21*. Half of the mice were sacrificed on Day 35* for analysis of the T cell response. Sera were prepared on Days 20, 35, 49, 63, 84, 105, 126, 147, 168 and 189*. Measles-specific IgG immune responses were monitored using an ELISA assay. The GMTs were expressed in mIU/ml (milli-International Units of anti-measles IgG were calculated using a human sera standard). Error bars showing ±SEM.

*Relative to the day of birth.

FIG. 22 depicts N-specific Cellular Immune in Newborn or 7 Days Old Mice Vaccinated Twice with MVA-mBN85B. Newborn or 7 days old BALB/c mice (n=6 per group) were vaccinated respectively on Days 0* or 7* s.c. with 1×10⁸ TCID₅₀ MVA-mBN85B and then boosted on Day 21*. The control mice received TBS on Days 7* and 21*. Splenocytes were prepared on Day 35* and stimulated with two N-specific peptides (peptide 1: YPALGLHEF (SEQ ID NO:5) and peptide 2: YAMGVGVELEN (SEQ ID NO:6). N-specific T cell immune responses were monitored using an IFN-γ ELISpot assay. Average Spot Forming Cells/10⁶ splenocytes ±SEM are shown.

*Relative to the day of birth.

FIG. 23 depicts the induction of anti-measles antibodies in mice with MVA-Measles vs Rouvax. All animals received either 2 doses MVA-Measles (1×10⁸ TCID₅₀) or the recommended dose of Rouvax.

FIG. 24 depicts Humoral Immune Response in Newborn or 7 Days Old Mice Vaccinated with MVA-mBN85B. Newborn or 7 days old BALB/c mice (n=5 or 6 per group) were vaccinated respectively on Day 0* or 7* s.c. with 1×10⁸TCID₅₀ MVA-mBN85B and then boosted or not on Day 21*. The control mice received TBS on Days 0* and 21*. Sera were prepared on Days 20, 35, 49, 63, 84, 105, 126, 147, 168 and 189*. Measles-specific IgG immune responses were monitored using an ELISA assay. The GMTs were expressed in mIU/ml (milli-International Units of anti-measles IgG were calculated using a human sera standard). Error bars showing ±SEM.

*Relative to the day of birth

FIG. 25 depicts the induction of anti-measles antibodies in humans with MVA-Measles vs Rouvax. All subjects received either 1 dose MVA-Measles (1×10⁸ TCID₅₀) or the recommended dose of Rouvax. A second immunization with MVA-Measles did not result in further increases in titers. The results show a 275% better response with MVA-Measles compared to Rouvax (Quartile 3 best approximates Rouvax preimmunization titers).

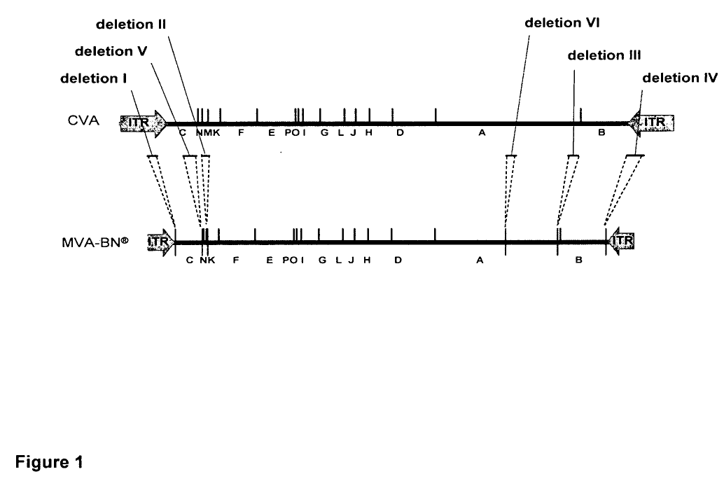

### EXAMPLES

### Origin of Inserted Genes

H (hemagglutinin protein), F (fusion protein) and N (nucleoprotein) coding sequences were amplified from RNA of the measles strain Khartoum SUD/34.97 (Genotype B3). RNA was transcribed by RT-PCR into cDNA (SuperscriptIII, Invitrogen).

Hemagglutinin (H) is a surface glycoprotein responsible for virus binding to suitable receptors on the host cells. The Fusion protein (F) is also on the surface and responsible for fusion of the viral envelope with the target cell membrane. H is an essential cofactor for promoting fusion and F and H together are responsible for immunosuppressive properties of the measles virus. Nucleoprotein (N) belongs to the structural proteins and is responsible for encapsidation of the measles genome (RNA).

cDNA of the H, F and N genes was inserted into a cloning vector (TOPO TA, Invitrogen) and sequenced. The H, F and N Gene Sequences from cDNA transcribed from RNA isolated from measles strain Khartoum SUD/34.97 (Genotype B3) are provided below. The H gene shows 99% homology to GenBank AF453-430 (Measles strain Khartoum.SUD/33.97 hemagglutinin (H) gene), the F gene shows 98% homology to GenBank AY059392 (Measles virus G954 fusion protein (F) mRNA), and the N gene shows 99% homology to GenBank AJ232771 (Measles virus RNA gene encoding nucleoprotein, isolate MVi/Lagos.NIE/11.98/1).

The H Gene Sequence (1854 bp):

(SEQ ID NO: 1)
atgtcaccgcaacgagaccggataaatgccttctacaaagataaccct
tatcccaagggaagtaggatagttattaacagagaacatcttatgatt
gacagaccctatgttttgctggctgttctgttcgtcatgtttctgagc
ttgatcgggttgctggccattgcaggcattagacttcatcgggcagcc
atctacaccgcggagatccataaaagcctcagtaccaatctagatgtg
actaactccatcgagcatcaggtcaaggacgtgctgacaccactcttt
aaaatcatcggggatgaagtgggcctgagaacacctcagagattcact
gacctagtgaaattcatctctgacaagattaaattccttaatccggat
agggagtacgacttcagagatctcacttggtgcattaacccgccagag
agaatcaaactggattatgatcaatactgtgcagatgtagctgctgaa
gagctcatgaatgcattggtgaactcaactctactggagaccagaaca
accaatcagttcctagctgtctcaaagggaaactgctcagggcccact
acaatcagaggtcaattctcaaacatgtcgctgtccttgttggacttg
tacttaggtcgaggttacaatgtgtcatctatagtcactatgacatcc
cagggaatgtatgggggaacctacctagtggaaaagcctaatctgaac
agcaaagggtcagagttgtcacaactgagcatgtaccgagtgtttgaa
gtaggtgttatcagaaacccgggtttgggggctccggtgttccatatg
acaaattattttgagcaaccagtcagtaatggtctcggcaactgtatg
gtggctttgggggagctcaaactcgcagccctttgtcacggggacgat
tctatcacaattccctatcagggatcagggaaaggtgtcagcttccag
ctcgtcaagctgggtgtctggaaatccccaaccgacatgcaatcctgg
gtctccttatcaacggatgatccagcggtagacaggctttacctctca
tctcacagaggtgtcatcgctgacaatcaagcaaaatgggctgtcccg
acaacacgaacagatgacaagctgcgaatggagacatgcttccagcag
gcgtgtaaaggtaaaatccaagcactctgcgagaatcccgagtgggca
ccattgaaggataacaggattccttcatacggggtcctgtctgttgat
ctgagtctgacggttgagcttaaaatcaaaattgcttcgggattcggg
ccattgatcacacacggctcagggatggacctatacaaatccaaccgc
aacaacgtgtattggctgactatcccgccaatgaggaatctagcctta
ggcgtaatcaacacattggagtggataccgagattcaaggttagtccc
aacctcttcactgtcccaattaaggaagcaggcgaagactgccatgcc
ccaacatacctacctgcggaagtggacggtgatgtcaaactcagttcc
aacctggtgatcctacctggtcaagatctccaatatgttttggcaacc
tacgatacttccagggttgagcatgctgtggtttattacgtttacagc
ccaagccgctcattttcttacttttatccttttaggttgcctataaag
ggggtcccaatcgaattacaagtggaatgcttcacatgggaccaaaaa
ctctggtgccgtcacttctgtgtgcttgcggactcagaatccggtgga
cttatcactcactctgggatggtaggcatgggagtcagctgcacagcc
acccgggaagatggaaccaaacgcagatag

F Gene Sequence (1653 bp):

(SEQ ID NO: 2)
atgggtctcaaggtggacgtctttgccatattcatggcagtactgtta
actctccaaacacccgccggtcaaatccattggggcaatctctctaag
ataggtgtagtaggaataggaagtgcaagctacaaagttatgactcgt
tccagccatcaatcattagtcataaaattaatgcccaatataactctc
ctcaataactgcacgagggtagagattgcagaatacaggagactacta
agaacagttttggaaccaattagagatgcacttaatgcaatgacccag
aacataaggccggttcagagcgtagcttcaagtaggagacacaagaga
tttgcgggagtagtcctggcaggtgcggccctaggtgttgccacagct
gctcagataacagccggcattgcgcttcaccagtccatgctgaactct
caggccatcgacaatctgagagcgagcctggaaactactaatcaggca
attgaggcaatcagacaagcagggcaggagatgatattggctgttcag
ggtgtccaagactacatcaataatgagctgataccgtctatgaaccaa
ctatcttgtgatttaatcggtcagaagctcgggctcaaattgctcaga
tactatacagaaatcctgtcattatttggccccagcctacgggacccc
atatctgcggagatatctatccaggctttgagctatgcacttggagga
gatatcaataaggtgttagaaaagctcggatacagtggaggcgattta
ctaggcatcctagagagcagaggaataaaggctcggataactcacgtc
gacacagagtcctacttcattgtcctcagtatagcctatccgacgctg
tccgagattaagggggtgattgtccaccggctagagggggtctcgtac
aacataggctctcaagagtggtataccactgtgcccaagtatgttgca
acccaagggtaccttatctcgaattttgatgagtcatcatgtactttc
atgccagaggggactgtgtgcagccaaaatgccttgtacccgatgagt
cctctgctccaagaatgcctccgagggtccaccaagtcctgtgctcgt
acactcgtatccgggtcttttgggaaccggttcattttatcacaaggg
aacctaatagccaattgtgcatcaattctttgtaagtgttacacaaca
ggaacgatcattaatcaagaccctgacaagatcctaacatacattgct
gccgatcgctgcccggtagtcgaggtgaacggcgtgaccatccaagtc
gggagcaggaggtatccagacgctgtgtacttgcacagaattgacctc
ggtcctcccatatcattggagaggttggacgtagggacaaatctgggg
aatgcaattgccaaattggaggatgccaaggaattgttggaatcatcg
gaccagatattgaggagtatgaaaggtttatcgagcactagcatagtc
tacatcctgattgcagtgtgtcttggagggttgatagggatccccact
ttaatatgttgctgcagggggcgttgtaacaaaaagggggaacaagtt
ggtatgtcaagaccaggcctaaagcctgaccttacaggaacatcaaaa
tcctatgtaaggtcgctttga

N Gene Sequence (1578 bp):

(SEQ ID NO: 3)
atggccacacttttgaggagcttagcattgttcaaaagaaacaaggac
aaaccacccattacatcaggatccggtggagccatcagaggaatcaaa
cacattattatagtaccaatccctggagattcctcaattaccactcga
tccagacttctggaccggttggtcaggttaattggaagcccggatgtg
agcgggcccaaactaacaggggcactaataggtatattatccttattt
gtggagtctccaggtcaattgattcagaggatcactgatgaccctgac
gttagcataaggctgttagaggttgtccagagcgaccagtcacaatct
ggccttaccttcgcatcaagaggtactaatatggagtatgaggcggac
cagtacttttcacatgatgatccaagtagtagtgatcaatccaggttc
gggtggtttgagaacaaggaaatctcagatattgaagtgcaagaccct
gagggcttcaacatgattctgggtaccatcctagctcaaatttgggtc
ttgctcgcaaaggcggttacggctcctgacacagcagctgattcggag
ctaagaaggtggatcaaatacacccaacaaagaagagtagttggtgaa
tttagattagagagaaaatggttggatgtggtgagaaacaggattgcc
gaggacctctccttacgccgattcatggtcgctctaatcctggatatc
aagaggacacccgggaacaaacccaggattgctgaaatgatatgtgac
attgatacatatatcgtagaggcaggattagccagttttatcctgact
attaagtttggaatagaaactatgtatcctgctcttggactgcatgaa
tttgctggtgaattatccacacttgagtccttgatgaatctttaccag
caaatgggggaaactgcaccctatatggtaatcctggagaactcaatt
cagaacaagtttagcgcaggatcataccctttgctctggagctatgcc
atgggagtaggagtggaacttgaaaactccatgggaggtttgaacttc
ggccgatcttactttgatccagcatattttagattagggcaagagatg
gtaaggaggtcagctggaaaggtcagttccacattggcatctgaactc
ggtatcactgccgaggatgcaaggcttgtttcagagattgcaatgcat
actactgaggacaggatcagtagagcagttggacccagacaagcccaa
gtgtcattcctacacggtgatcaaagtgagaatgagctgccgagattg
gggggcaaggaggacaggagggtcaaacagagccgaggagaagccggg
gagagccacagagaaaccgggcccagcagagcaagtgatgcgagagct
gcccatcctccaaccggcacacccctagacattgacactgcatcggag
ttcagccaagatccgcaggacagtcgaaggtcagccgatgccctgctt
aggctgcaagccatggcaggaatctcggaagaacaagactcagacacg
gacacccctagagtgtacaatgacagagatcttctagactag

### Origin of Inserted Promoter

The A-type inclusion body promoter of cowpox virus (ATI), a late promoter, was synthetically generated and cloned into the plasmid pBluescript KS+ (Stratagene) resulting in plasmid pBNX65. The ATI promoter was inserted in front of the H, F and N sequence. Consequently, the H, F and N proteins should be expressed with other late genes, after DNA replication. The sequence of the ATI Promoter is:

| | |
|---|---|
| GTTTTGAATAAAATTTTTTTATAATAAATC. | (SEQ ID NO: 4) |

### Origin and Construction of Recombination Plasmids pBNX86, pBNX87 and pBNX118

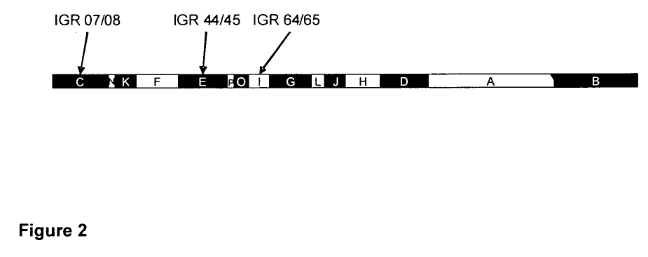

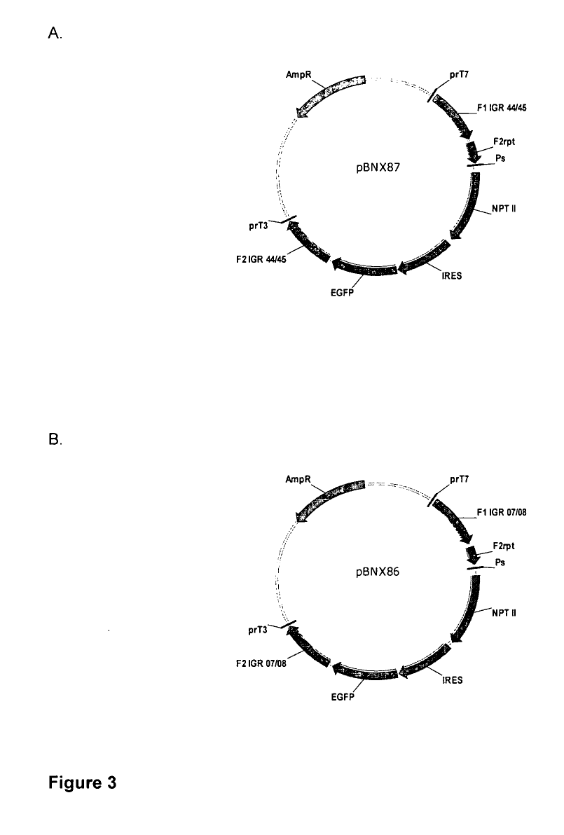

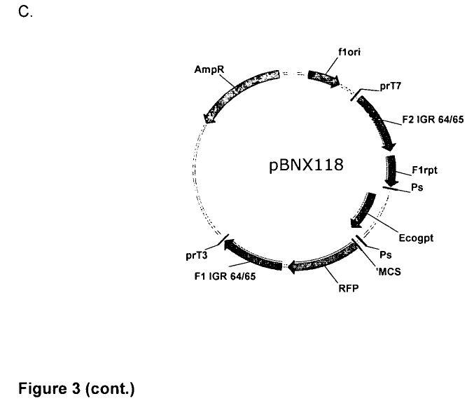

For the insertion of foreign genes into the MVA-BN® genome several recombination plasmids were constructed that target the intergenic (non-coding) regions (IGR)(FIG. 2). pBNX86, pBNX87 and pBNX118 (FIG. 3) are plasmids containing MVA-BN® DNA sequences from the regions that flank the IGR between the open reading frames (ORF) ORF 64 and 65 (IGR 64/65; pBNX118), between the ORF 07 and 08 (IGR 07/08; pBNX86) and between the ORF 44 and 45 (IGR 44/45; pBNX87). For generation of recombinant MVA-BN® products, foreign sequences of interest (e.g. F, H, or N) can be inserted into a suitable recombination plasmid targeting these intergenic regions.

After infection of CEF cells with MVA-BN® and subsequent transfection with the appropriate recombination plasmid, homologous recombination of the plasmid flanking sequences with the homologous sequences of the MVA-BN® virus targets the insertion of the plasmid sequences into the respective site (e.g. IGR) of the MVA-BN® genome (FIG. 2). The presence of a selection cassette in the inserted sequences allows for positive selection of recombinant MVA-BN® viruses.

pBNX86

MVA-BN® DNA sequences flanking the intergenic region between the ORF 07 and 08 (flank1, F1 and flank 2, F2 and a sequence repeat of F2, F2rpt) in the HindIII fragment of the MVA-BN® genome were amplified and cloned into pBluescript KS+. The sequence repeat of flank 2 (F2rpt) was inserted to mediate deletion of the selection cassette after isolation of recombinant viruses. Between flank F2 and F2 repeat of IGR 07/08, the coding sequence for the neomycin resistance gene (NPTII) was inserted under the control of a strong synthetic Vaccinia virus promoter (Ps) followed by an internal ribosomal entry site (IRES) and the enhanced green fluorescence protein gene (EGFP). This resulted in a bicistronic expression cassette for NPTII and EGFP in the plasmid designated pBNX86 (FIG. 3).

pBNX87

MVA-BN® DNA sequences flanking the intergenic region between the ORF 44 and 45 (flank1, F1, and flank2, F2, and a sequence repeat of flank2, F2rpt) in the HindIII fragment of the MVA-BN® genome were amplified and cloned into pBluescript KS+. The sequence repeat of flank 2 (F2rpt) was inserted to mediate deletion of the selection cassette after isolation of the recombinant viruses. Between flank F2 and F2 repeat of IGR 44/45, the coding sequence for the neomycin resistance gene (NPTII) was inserted under the control of a strong synthetic Vaccinia virus promoter (Ps) resulting in an intermediate plasmid (not shown). After the NPTII gene, an internal ribosomal entry site (IRES) and the enhanced green fluorescence protein gene (EGFP) were inserted resulting in a bicistronic expression cassette for NPTII and EGFP.

pBNX118

MVA-BN® DNA sequences flanking the intergenic region between the ORF 64 and 65 (flank1, F1, and flank2, F2, and a sequence repeat of flank1, F1rpt) in the HindIII fragment of the MVA-BN® genome were amplified and cloned into pBluescript KS+. The sequence repeat of flank 1 (F1rpt) was inserted to mediate deletion of the selection cassette after isolation of the recombinant viruses. Between flank F1 and F1 repeat of IGR 64/65, the coding sequence for the E. coli gpt drug selection gene (Ecogpt) was inserted under the control of a strong synthetic Vaccinia virus promoter (Ps) resulting in an intermediate plasmid. After the gpt gene a red fluorescence protein gene (RFP) was inserted also under the control of the strong synthetic Vaccinia virus promoter (Ps) resulting in a bicistronic expression cassette for gpt and RFP.

### Cloning of the Final Recombination Plasmid pBN133

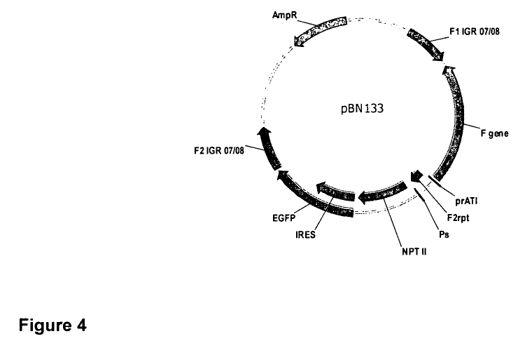

To create recombinant MVA-mBN85B, the final recombination plasmid pBN133 (FIG. 4) was constructed by inserting the F gene into the recombination plasmid pBNX86. Therefore, the F gene was inserted in the promoter vector pBNX65—resulting in pBN132. In the next step, the promoter together with the F gene were inserted in pBNX86—resulting in pBN133. In summary, pBN133 contains the F gene under the control of the cowpox virus ATI promoter and a selection cassette (NPTII and EGFP) under the control of the strong synthetic vaccinia virus promoter Ps. In addition, pBN133 contains MVA-BN® DNA sequences that flank the IGR 07/08 within the MVA-BN® genome and a sequence repeat of flank 2 to allow the later elimination of the selection cassette by homologous recombination.

### Cloning of the Final Recombination Plasmid pBN135

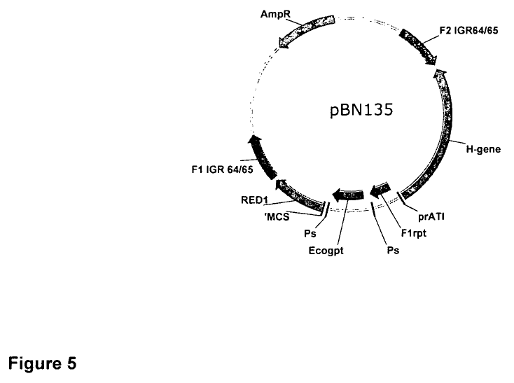

To create recombinant MVA-mBN85B, the final recombination plasmid pBN135 (FIG. 5) was constructed by inserting the H gene into the recombination plasmid pBNX118. Therefore, the H gene was inserted in the promoter vector pBNX65—resulting in pBN134. In the next step, the promoter together with the H gene were inserted in pBNX118—resulting in pBN135. In summary, pBN135 contains the H gene under the control of the cowpox virus ATI promoter and a selection cassette (Ecogpt and RFP (RED)) under the control of the strong synthetic vaccinia virus promoter Ps. In addition, pBN135 contains MVA-BN® DNA sequences that flank the IGR 64/65 within the MVA-BN® genome and a sequence repeat of flank 1 to allow the later elimination of the selection cassette by homologous recombination.

### Cloning of the Final Recombination Plasmid pBN157

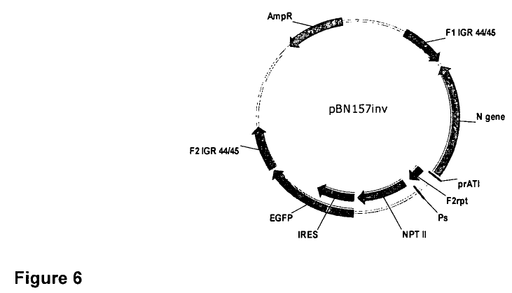

To create recombinant MVA-mBN85B, the final recombination plasmid pBN157 (FIG. 6) was constructed by inserting the N gene into the recombination plasmid pBNX87. Therefore, the N gene was inserted in the promoter vector pBNX65—resulting in pBN155. In the next step the promoter together with the N gene were inserted in pBNX87—resulting in pBN157. In summary, pBN157 contains the N gene under the control of the cowpox virus ATI promoter and a selection cassette (NPTII and EGFP) under the control of the strong synthetic vaccinia virus promoter Ps. In addition, pBN157 contains MVA-BN® DNA sequences that flank the IGR 44/45 in the MVA-BN® genome and a sequence repeat of flank 2 to allow the later elimination of the Ps selection cassette by homologous recombination.

### Generation of Recombinant MVA-mBN85B

To create a recombinant vaccinia vector expressing the H, F, and N proteins of the measles virus, the final recombination plasmids pBN133, pBN135 and pBN157 were created, as described in the previous section. Primary chicken embryo fibroblast (CEF) cells were infected with MVA-BN® (passage 584) and subsequently transfected with pBN133. The resulting intermediate recombinant MVA-BN® product designated MVA-mBN68 containing the F gene coding region and the selection cassette was obtained after multiple (4) plaque purifications under selective conditions and amplified.

Subsequently, primary CEF cells were infected with MVA-mBN68 and transfected with pBN135. The resulting intermediate double recombinant MVA-BN® product was designated MVA-mBN75A. MVA-mBN75A contains the F and H gene coding regions and the selection cassette. It was obtained after multiple (5) plaque purifications under selective conditions. After amplification and further plaque purifications (2) under non-selective conditions the intermediate recombinant virus MVA-mBN75B partly devoid of the selection cassette was isolated—meaning no fluorescence was visible under the microscope any longer.

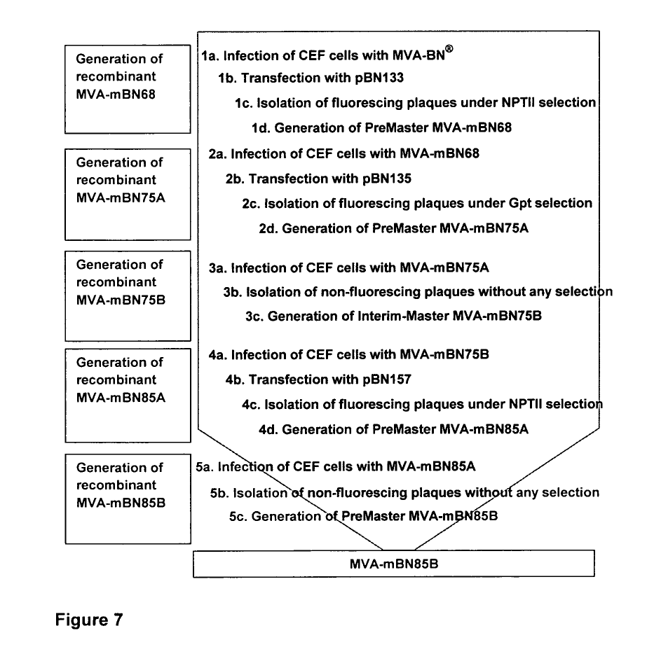

Subsequently, primary CEF cells were infected with MVA-mBN75B and transfected with pBN157. The intermediate triple recombinant MVA-BN® virus product was designated MVA-mBN85A. It contains the F, H, and N gene coding regions and the selection cassette and was obtained after multiple (5) plaque purifications under selective conditions. After amplification and further plaque purification under non-selective conditions the recombinant MVA-BN® product MVA-mBN85B devoid of the selection cassettes was isolated. In total, 60 passages were involved in the generation of the MVA-mBN85B PreMaster, of which 21 passages were plaque purifications. At all stages, VP-SFM serum-free medium was used. The generation of MVA-mBN85B is summarized in FIG. 7.

### Characterization of MVA-mBN85B PreMaster Virus Stocks

Different MVA-mBN85B PreMaster virus stocks were established (PP4, PP5 and PP6, as well as different clones from PP5) and examined for elimination of the MVA-BN® empty vector virus, for elimination of the selection cassette, for sterility and for correct size of the insert. Additionally, the titer of the MVA-mBN85B PreMaster virus stock was determined. Sequence was not determined on the PreMaster, but on the subsequently produced Master Virus Bank (MVB) from MVA-mBN85B. The sequence for the MVB was 100% identical with the expected sequence. The MVA-mBN85B PreMaster virus stock (PP5, clone #6) was finally used for Master Virus Bank (MVB) production.

### Identity of MVA-mBN85B

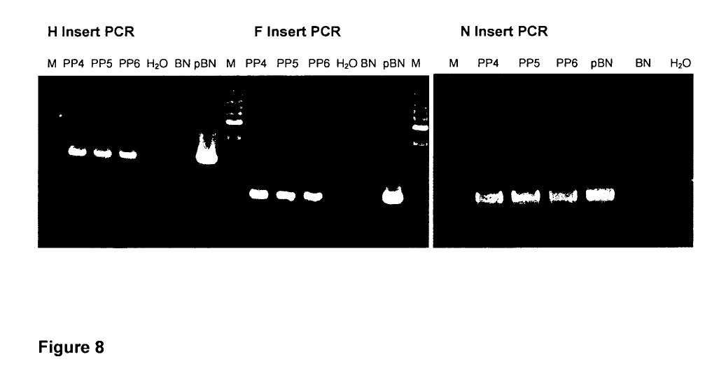

The correct size of the inserted genes in IGR 07/08, IGR 44/45, and IGR 64/65 were confirmed by a gene specific PCR amplifying the F, H and N genes (FIG. 8) using the primers in Table 3. The sequence of F, H, and N in the MVA-mBN85B PP5 PreMaster was not determined, since the correct coding sequence had been determined for plasmid pBN133, pBN135, and pBN157. Sequencing was performed for the MVB. The sequence analysis revealed a 100% homology to the predicted sequence.

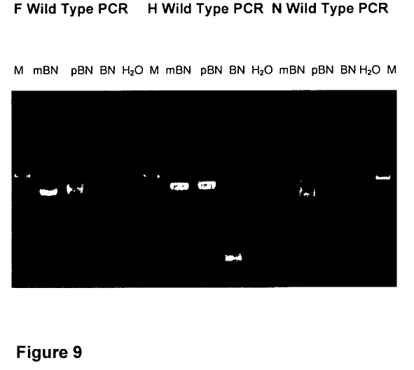

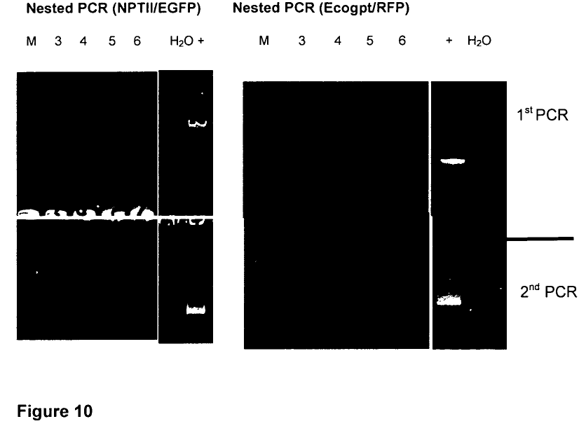

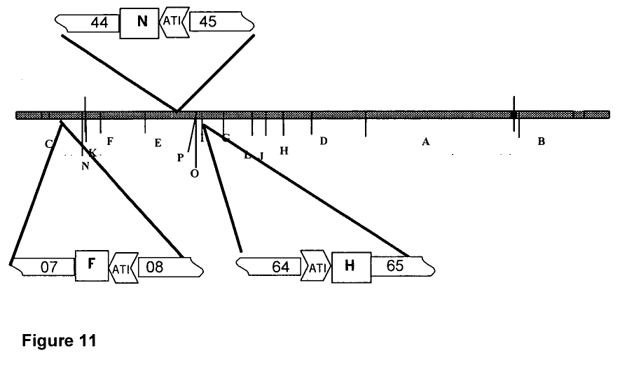

The absence of MVA-BN® empty virus vector and the correct insertion of the F, H and N gene into to the IGR 07/08, IGR 44/45 and IGR 64/65 was confirmed by IGR 07/08, IGR 44/45 and IGR 64/65 specific PCR on PreMaster of the MVA-mBN68 for the F gene, of the MVA-mBN75A for the H gene, and of the MVA-mBN85A for the N gene (FIG. 9). PreMaster MVA-mBN85B was not separately tested on all the inserts, since a sequencing was performed, which showed that the genes F, H, and N are inserted correctly and that the sequence is correct. The absence of the selection cassette used during generation of the recombinant MVA-mBN85B virus was confirmed by nested PCR analysis (FIG. 10). A schematic map of the genomic part of MVA-mBN85B virus is shown in FIG. 11.

TABLE 3
Primer Sequences

| | | | | Position | |
|---|---|---|---|---|---|
| Primer | | | | in the | Used |
| No. | Purpose | Orientation | Prime Sequence: 5′-3′ | plasmid | with |
| 902 | Wildtype PCR flank 07/08 | sense | CTGGATAAATACGAGGACGTG | 1124 | 903: |
| | | | (SEQ ID NO: 5) | | 1988 |
| | | | | | bp |
| 903 | Wildtype PCR flank 07/08 | antisense | GACAATTATCCGACGCACCG | 3112 | |
| | | | (SEQ ID NO: 6) | | |
| 499 | Wildtype PCR flank 64/65 | antisense | CAACTCTCTTCTTGATTACC | 3380 | 500: |
| | | | (SEQ ID NO: 7) | | 2333 |
| | | | | | bp |
| 500 | Wildtype PCR flank 64/65 | sense | CGATCAAAGTCAATCTATG | 1047 | |
| | | | (SEQ ID NO: 8) | | |
| 904 | Wildtype PCR flank 44/45 | sense | CGTTAGACAACACACCGACGATGG | 1169 | 905: |
| | | | (SEQ ID NO: 9) | | 1906 |
| | | | | | bp |
| 905 | Wildtype PCR flank 44/45 | antisense | CGGATGAAAAATTTTTGGAAG | 3075 | |
| | | | (SEQ ID NO: 10) | | |

### Functionality of MVA-mBN85B

Reverse Transcriptase PCR (RT-PCR) was performed. A clear band of transcribed mRNA was found for F, H and N. No bands were found in the negative controls: MVA-mBN85B without the enzyme reverse transcriptase and for the MVA-BN® with and without reverse transcriptase. Sterility testing was performed. No microbial growth was observed. Titration of MVA-mBN85B revealed a virus titer of 7.5×10⁶ TCID₅₀/ml.

### Safety Evaluation of MVA-mBN85B in Male and Female Adult Rats

The toxicity and local tolerance of MVA-mBN85B was investigated in adult Sprague-Dawley rats (aged approximately 9 weeks at the first administration) following two administrations (s.c.) of either 1×10⁸ TCID₅₀ of MVA-mBN85B or TBS as control vehicle in a four week interval (Day 1 and 29). The reversibility of any observations was assessed by having either a 2 day or a 28 day treatment free period. Half of the study animals were necropsied after these two periods (see Table 4).

TABLE 4
Summary of Study Design

| | | | Number of Animals^{b} | |
|---|---|---|---|---|
| | | | 2 Day Free | |
| Test or | | Day of | Treatment | 28 Day Free |
| Reference Item^{a} | Route | Admin. | Period | Treatment Period |
| TRIS-buffered Saline | s.c. | 1, 29 | 5M + 5F | 5M + 5F |
| (TBS) | | | | |
| 1 × 10⁸ TCID₅₀ | s.c. | 1, 29 | 5M + 5F | 5M + 5F |
| MVA-mBN85B | | | | |

^{a}Nominal titer;
^{b}Male (M) or female (F)

All animals survived until their scheduled necropsy. No vaccine-related effects on body weight, food consumption, gross pathology, organ weights, urinalysis, and ophthalmology were observed. This study demonstrated that two administrations (s.c.) of MVA-mBN85B at 1×10⁸ TCID₅₀ in adult rats was associated with some minimal, yet transient injection site reactions and microscopic findings. However, due to the reversible nature of these findings, MVA-BN85B can be considered non-toxic and safe.

### Safety Evaluation of MVA-mBN85B in Male and Female Juvenile Rats

The toxicity and local tolerance of MVA-mBN85B was investigated in juvenile Sprague-Dawley rats following three administrations (s.c.) of MVA-mBN85B (either 1×10⁷ or 1×10⁸ TCID₅₀) or TBS as control at weekly intervals on post-natal days (PND) 21, 28, and 35. The reversibility of any observations was assessed after a 2 and a 28 day treatment free period with half of the animals being necropsied after these two periods and tissue samples prepared for analysis (see Table 5).

TABLE 5
Summary of Study Design

| | | | Number of Animals^{b} | |
|---|---|---|---|---|
| | | | 2 Day Free | 28 Day Free |
| Test or Reference | | Day of | Treatment | Treatment |
| Item^{a} | Route | Admin. | Period | Period |
| TRIS-buffered Saline | s.c. | PND^{b} | 5M + 5F | 5M + 5F |
| (TBS) | | 21, 28, 35 | | |
| 1 × 10⁷ TCID₅₀ | s.c. | PND | 5M + 5F | 5M + 5F |
| MVA-mBN85B | | 21, 28, 35 | | |
| 1 × 10⁸ TCID₅₀ | s.c. | PND | 5M + 5F | 5M + 5F |
| MVA-mBN85B | | 21, 28, 35 | | |

^{a}Nominal titer;
^{b}PND = post-natal day;
^{c}Male (M) or female (F)

All animals survived until their scheduled necropsy. No vaccine-related effects on body weight, food consumption, organ weights, urinalysis, and ophthalmology were observed. In summary, subcutaneous injection of 1×10⁷ or 1×10⁸ TCID₅₀ MVA-mBN85B on Days 21, 28 and 35 days of age was well tolerated by the juvenile rat; there were only minor incidences of reddening of the skin at the injection sites noted at necropsy. There was no systemic toxicity or local irritation.

### MVA-mBN85B Fails to Reproductively Replicate in Human Cells

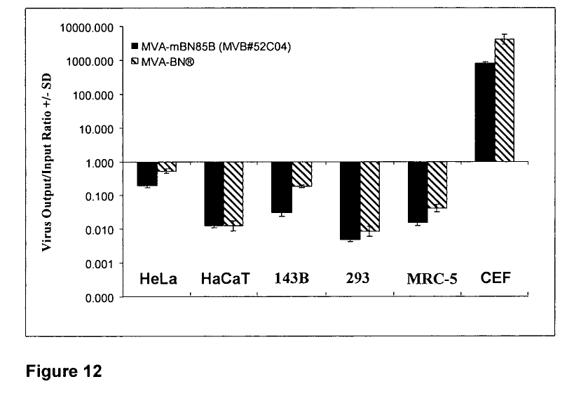

MVA-BN® has been demonstrated to have a superior attenuation profile compared to other MVA isolates and that it fails to reproductively replicate in human cell lines (WO 02/42480). To ensure that the insertion of the three measles genes into the MVA-BN® genome has not altered the attenuation of MVA-mBN85B, the ability of this recombinant virus to reproductively replicate in a variety of human cell lines was investigated and compared to MVA-BN®. As shown in FIG. 12, MVA-mBN85B and MVA-BN® only reproductively replicated in CEF cells, the primary cells used to produce the vaccines. Importantly, MVA-mBN85B had an identical replication profile as MVA-BN® and both viruses failed to reproductively replicate in any of the human cell lines evaluated including HeLa (cervical cancer cell line), HaCaT (keratinocyte cell line), 143B (bone osteosarcoma cell line), 293 (kidney cell line), or MRC-5 (embryonic lung cell line).

### MVA-mBN85B Lacks the Ability to Cause Cell Fusion

As MVA-mBN85B encodes the F gene from measles, a protein known to induce cell fusion, studies were conducted to examine whether MVA-mBN85B retained this property normally displayed by viruses like measles that belong to the Paramyxoviridae family. Briefly, different human cell lines (TF-1, HeLa and HUVEC) were inoculated with MVA-mBN85B and examined after 4 days for the presence of multi-nucleated cells, indicating cell fusion, using the Hoechst staining technique. As a positive control Sendai virus, a member of the Paramyxoviridae was used, which induced cell fusion (>10%) in all the human cells tested. In contrast, MVA-mBN85B demonstrated similar levels (<1%) of cell fusion as the negative control (assay media only), clearly demonstrating that MVA-mBN85B lacked the potentially toxic property of inducing cell fusion.

### Immunological Studies of MVA-mBN85B in Adult Rats

To initially assess the immunogenicity of MVA-mBN85B, anti-measles IgG titers were measured in the sera obtained from the toxicity study in adult rats. Briefly, adult Sprague-Dawley rats were administered (s.c.) either MVA-mBN85B (1×10⁸ TCID₅₀) or TBS as control on Days 1 and 29. Blood collected from these rats on Day −1 (pre-vaccination) and on Day 57 was used to investigate the humoral immune response by ELISA.

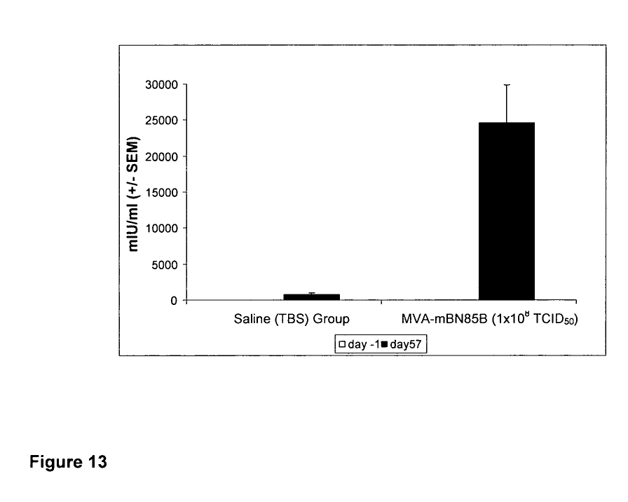

As illustrated in FIG. 13, there was a clear induction of measles-specific IgG antibodies (24,549 mIU/ml ±5,380 SEM) in serum samples collected from the rats vaccinated with MVA-mBN85B. All MVA-mBN85B vaccinated animals (n=10) maintained in the study until Day 57 had seroconverted.

### Immunological Response of Three Different Doses of MVA-mBN85B in Adult Mice

Two different dose response studies were conducted. In addition to the ELISA used to assess antibody responses, N-specific T cell responses were measured by an IFN-γ ELISpot assay after stimulation of splenocytes with two different N-specific peptides, peptide 1: YPALGLHEF (SEQ ID NO:11) and peptide 2: YAMGVGVELEN (SEQ ID NO:12). Concanavalin A, a lectin stimulating T cells, was always used as positive control and resulted in IFN-γ responses by splenocytes from each single mouse.

### Humoral Immune Responses Induced by MVA-mBN85B

BALB/c mice were immunized four times in three week intervals with 1×10⁶, 1×10⁷, or 1×10⁸ TCID₅₀ MVA-mBN85B. ELISA was performed on serum samples from blood collected on Days -1, 20, 41, 62, and 77 as described in Table 6.

TABLE 6
Summary of Study Design

| Test or Reference Item Administration | | | Bleed | Necropsy |
|---|---|---|---|---|
| | Dose | Schedule | Schedule | Schedule |
| Name | (TCID₅₀) | (Day*) | (Day*) | (Day*) |
| TBS | — | 0, 21, 42, 63 | −1, 20, 41, 62, 77 | 77 |
| MVA-BN ® | 1 × 10⁸ | 0, 21, 42, 63 | −1, 20, 41, 62, 77 | 77 |
| MVA- | 1 × 10⁶ | 0, 21, 42, 63 | −1, 20, 41, 62, 77 | 77 |
| mBN85B | | | | |
| MVA- | 1 × 10⁷ | 0, 21, 42, 63 | −1, 20, 41, 62, 77 | 77 |
| mBN85B | | | | |
| MVA- | 1 × 10⁸ | 0, 21, 42, 63 | −1, 20, 41, 62, 77 | 77 |
| mBN85B | | | | |

*Relative to first immunization

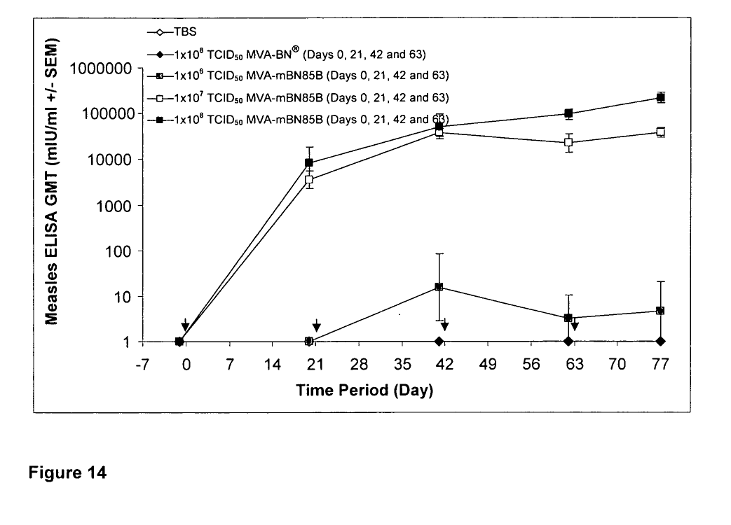

As depicted in FIG. 14, high levels of antibody titers were readily detectable in all mice 20 days after the first administration of 1×10⁷ TCID₅₀ MVA-mBN85B. These were almost equivalent to the higher dose of 1×10⁸ TCID₅₀. A boost effect was detected following the second administration (6-fold), whereas measles-specific antibody responses did not increase much further after the third and the fourth administration. The lower dose of 1×10⁶ TCID₅₀ MVA-mBN85B induced minor antibody responses only, with a seroconversion rate of 40%. As expected, no measles-specific humoral response was observed in mice immunized with MVA-BN®, the viral vector without measles inserts or TBS.

BALB/c mice were immunized twice in a three week interval with 1×10⁶, 1×10⁷, or 1×10⁸ TCID₅₀ MVA-mBN85B. ELISA was performed on day 20 and 35. This second dose response study confirmed previous results. As indicated in Table 7, all mice immunized twice on Day 0 and Day 21 with 1×10⁷ or 1×10⁸ TCID₅₀ MVA-mBN85B showed similar titers as above, with incomplete seroconversion (50%) in the low dose group, i.e. 1×10⁶ TCID₅₀.

TABLE 7
Summary of Study Design

| Test or Reference Item Administration | | | Bleed | Necropsy |
|---|---|---|---|---|
| | Dose | Schedule | Schedule | Schedule |
| Name | (TCID₅₀) | (Day*) | (Day*) | (Day*) |
| TBS | — | 0, 21 | 20, 35 | 35 |
| MVA-mBN85B | 1 × 10⁶ | 0, 21 | 20, 35 | 35 |
| MVA-mBN85B | 1 × 10⁷ | 0, 21 | 20, 35 | 35 |
| MVA-mBN85B | 1 × 10⁸ | 0, 21 | 20, 35 | 35 |

*Relative to first immunization.

### Immunological Response of MVA-mBN85B in Mice

Individual blood samples were collected by retro-orbital puncture from all animals for sera IgG analysis. On Days 0, 21, 42, and 63 mice were administered subcutaneously (s.c.) with 500 μl of either TBS (Group 1) as negative control, MVA-BN® (Group 2) as MVA vector control not expressing the measles specific proteins, 10⁶ TCID₅₀ of MVA-mBN85B (Group 3), 10⁷ TCID₅₀ of MVA-mBN85B (Group 4). or 10⁸ TCID₅₀ of MVA-mBN85B (Group 5). As positive control, commercially available measles vaccine Merieux® was administered s.c. once on Day 0 (single administration is recommended in humans). An additional group of mice (Group 7) was administered s.c. with MVA-mBN85B on Day 63 to investigate whether it is possible to induce a cellular immune response by single administration of the vaccine (compared to four administrations in Group 5).

One day before the administrations (i.e. on Days −1, 20, 41, and 62), body weights from all animals were determined and blood samples from all animals were collected (on Days −1 and 20 by retroorbital puncture and on Days 41 and 62 by tail vein puncture). Body weight was also determined in weekly intervals between the administration (i.e. on Days 6, 13, 27, 34, 48, 55) and between the last administration and the sacrifice (i.e. on Days 69 and 76). On day 77, mice were finally bled retroorbitally and sacrificed by cervical dislocation. Following sacrifice, spleens were removed aseptically from each animal for subsequent analysis of cellular responses by IFNγ-ELISpot assay.

Analysis of Measles-Specific Antibody Titers from Serum Samples

The measles-specific IgG ELISA titers were determined from all serum samples with the “Enzygnost®” ELISA kit (Dade Behring, Ref.: OWLN15). This ELISA kit uses measles virus strain Edmonston (ATCC number: VR24™) and was modified as follows. Instead of peroxidase (POD) conjugated anti-human F(ab) fragments of an rabbit antibody (supplied with the kit), a horse radish peroxidase (HRP)-conjugated sheep anti-mouse IgG (from Serotec, Cat. No.: AAC10P) was used as a secondary antibody. Furthermore, 5 μl serum was diluted in 100 μl sample buffer.

Analysis of Measles N-Protein-Specific Cellular Responses from Splenocytes

Specific cellular immune responses were investigated by stimulation of splenocytes with potential measles N-protein specific peptides and detection of the released IFN-γ by an ELISpot assay. Briefly, spleens from individual mice were transferred into 25 ml Dispomix® tubes containing 10 ml refrigerated RPMI-10 medium (consisting of RPMI-1640 medium supplemented with 10% FBS, Penicillin/Streptomycin and β-Mercaptoethanol) and were homogenized using a Dispomix® device (program “Saw 03”). Following homogenisation, the cell numbers per spleen were manually counted from an aliquot using a Turks solution, a Madaus counting chamber and a light microscope. Each splenocyte suspension was adjusted to 5×10⁶ cells/ml and 5×10⁵, 2.5×10⁵, and 1.25×10⁵ cells/well were transferred in duplicates by serial dilution into ELISpot plates precoated with anti-IFNγ antibody. The duplicate incubations were stimulated either with the four N-protein specific peptides at a final concentration of 5 μg/ml or with a peptide pool containing the four peptides at the same concentration for each peptide.

As positive control, splenocytes were stimulated with staphylococcus enterotoxin B (SEB; from Sigma; Catalogue number: S4881) at a final concentration of 0.5 μg/ml. In addition, splenocytes were stimulated with MVA-BN® (6.19×10⁸ TCID₅₀/ml) having a Multiplicity of Infection (MOI) of 12 (i.e. 12 TCID₅₀/cell) for demonstrating proper subcutaneous administration of the MVA-derived products.

As negative control, cell suspensions were incubated with medium only. Following a stimulation period of approximately 19 hours, cells were washed from the ELISpot plates and they were further processed as recommended by the manufacturer (BD, Material number: 551083). In a final step the plates were incubated for approximately 25 minutes with AEC substrate reagent (BD, Catalogue number: 551951) for visualization of the individual spots.

### Data Processing and Evaluation

Mean body weight changes (in %) of each group were calculated in an Excel file for each monitored time point following normalization of the individual values using the body weight (in grams) prior to the first administration as baseline value. The body weight value prior to the first administration was normalized to be 100%. From the normalized individual body weight changes the group means and the standard error of the means (S.E.M.) were calculated in Excel. The individual ELISA titers were calculated by means of using the human reference serum included in the ELISA kit (since a mouse positive reference serum was not available). Individual ELISA titers below the “calculation limit” where arbitrarily assigned as 1 (with a corresponding Log10 value of 0). These individual “quantitative” ELISA titers were used to determine group means plus standard error of the means (S.E.M.) using Excel. The ELISpot plates were evaluated with a Zeiss Imaging System. The number of spot forming cells (SFC) was determined for each well. These numbers were transferred into an Excel file for further evaluation. From the incubation with 5×10⁵ cells/well, the mean numbers of the duplicate incubations (recalculated for a million splenocytes) were determined from each mouse. A corrected mean value was generated by subtracting the mean values of the medium incubation. In case the corrected mean value was below 0, these values were adjusted to be 0. Following determination of the frequency of IFN-γ releasing splenocytes from individual animals, the mean frequency plus standard error of the mean (S.E.M.) was calculated per group.

Determination of the humoral immune responses by ELISA

In this study, 5 animals per group were subcutaneously (s.c.) administered four times in a 3-week interval either with TBS (Group 1), 10⁶ TCID₅₀ MVA-mBN85B (Group 3), 10⁷ TCID₅₀ MVA-mBN85B (Group 4), or 10⁸ TC TCID₅₀ ID50 MVA-mBN85B (Group 5). A group of 5 mice was s.c. administered four times in a 3-week interval with 10⁸ TCID₅₀ MVA-BN® (Group 2), the vaccine backbone vector not containing the measles specific inserts. Another group of 5 mice was s.c. administered once (on Day 0) with the commercially available Measles vaccine Merieux® (from Sanofi-Pasteur). A final group was included into the study for evaluating the cellular immune responses following a single administration of MVA-mBN85B. Although this group was not of major importance for evaluating humoral responses, animals from this group were bled 14 days after the single administration of the vaccine and the serum samples were analyzed as well. Prior to the first administration, one day before the subsequent administrations, and on the day of necropsy, animals were bled and serum was prepared for subsequent analysis of Measles-specific IgG antibody titers from the individual serum samples. As shown in Table 8, the Measles-specific mean IgG antibody titers collected prior to the first administration were below the detection limit of the assay in all groups.

Table 8 shows the kinetics of measles specific IgG titers following subcutaneous administration of MVA-mBN85B, Measles vaccine Merieux®, MVA-BN®, or TBS. Prior to the first administration (on Day 0) or at the indicated time points relative to the first administration, blood samples were collected, processed to serum and analyzed for measles specific IgG responses with a commercially available kit from Dade Bering. The values are “quantitatively” calculated using the human reference provided in the kit. Values below the “calculation limit” were arbitrarily assigned a Log10 titre of 2.00 (for calculation purposes).

TABLE 8
Measles specific IgG titers

| | | | | Group 2 | | | Group 3 | | | Group 4 | | |
|---|---|---|---|---|---|---|---|---|---|---|---|---|
| | Group 1 | | | 10⁸ TCID₅₀ | | | 10⁶ TCID₅₀ | | | 10⁷ TCID₅₀ | | |
| | TBS | | | MVA-BN | | | MVA-mBN85B | | | MVA-mBN85B | | |
| | (Administrations on | | | (Administrations on | | | (Administrations on | | | (Administrations on | | |
| | Days 0, 21, 42, 63) | | | Days 0, 21, 42, 63) | | | Days 0, 21, 42, 63) | | | Days 0, 21, 42, 63) | | |
| Time period* | Group | | | Group | | | Group | | | Group | | |
| (in Days) | mean | S.E.M. | N | mean | S.E.M. | N | mean | S.E.M. | N | mean | S.E.M. | N |
| −1 | 0.00 | 0.00 | 5 | 0.00 | 0.00 | 5 | 0.00 | 0.00 | 5 | 0.00 | 0.00 | 5 |
| 20 | 0.00 | 0.00 | 5 | 0.00 | 0.00 | 5 | 0.00 | 0.00 | 5 | 3.55 | 0.19 | 5 |
| 41 | 0.00 | 0.00 | 5 | 0.00 | 0.00 | 5 | 1.18 | 0.73 | 5 | 4.58 | 0.08 | 5 |
| 62 | 0.00 | 0.00 | 5 | 0.00 | 0.00 | 5 | 0.50 | 0.50 | 5 | 4.35 | 0.21 | 5 |
| 77 | 0.00 | 0.00 | 5 | 0.00 | 0.00 | 5 | 0.66 | 0.66 | 5 | 4.58 | 0.11 | 5 |

| | Group 5 | | | Group 6 | | | Group 7 | | |
|---|---|---|---|---|---|---|---|---|---|
| | 10⁸ TCID₅₀ | | | Measles vaccine | | | 10⁸ TCID₅₀ | | |
| | MVA-mBN85B (4x) | | | Merieux ® | | | MVA-mBN85B (1x) | | |
| | (Administrations on | | | (Administration on | | | (Administration on | | |
| | Days 0, 21, 42, 63) | | | Day 0 only) | | | Day 63 only) | | |
| Time period | Group | | | Group | | | Group | | |
| (in Days*) | mean | S.E.M. | N | mean | S.E.M. | N | mean | S.E.M. | N |
| −1 | 0.00 | 0.00 | 5 | 0.00 | 0.00 | 0 | — | — | — |
| 20 | 3.92 | 0.35 | 5 | 1.02 | 0.63 | 0 | — | — | — |
| 41 | 4.71 | 0.28 | 5 | 1.26 | 0.80 | 0 | — | — | — |
| 62 | 4.98 | 0.11 | 5 | 0.74 | 0.74 | 0 | 0.00 | 0.00 | 0 |
| 77 | 5.34 | 0.11 | 5 | 0.73 | 0.73 | 0 | 3.88 | 0.13 | 0 |

Similarly, all other time points investigated in the TBS treated group (i.e. Group 1) or the MVA-BN® vaccinated group (i.e. Group 2) were found to be negative. All animals demonstrated a Measles-specific antibody response 20 days after the first administration of either 10⁷ or 10⁸ TCID₅₀ of MVA-mBN85B resulting in Log₁₀ titers of 3.55 or 3.92 in Groups 4 or 5, respectively. At this time point, the Measles-specific IgG response was below the detection limit in all animals administered with 10⁶ TCID₅₀ of MVA-mBN85B (i.e. in Group 3). In case of the group administered with Measles vaccine Merieux®, two out of five animals demonstrated detectable Measles-specific IgG antibody titers resulting in a mean Log₁₀ titre of 1.02 at this time point.

Booster vaccinations with 10⁸ TCID₅₀ of MVA-mBN85B on Days 21, 42, and 63 resulted in increased mean Measles-specific IgG titers of 4.71, 4.98, and 5.34, respectively. With a ten-fold lower dose of MVA-mBN85B, an increase of the mean Measles-specific IgG titre was achieved following the second administration of the vaccine with a Log₁₀ titre of 4.58 determined one day before the third administration.

The mean specific titers reached a plateau thereafter in this group. In case of the lowest dose of MVA-mBN85B investigated in this study, two out of five animals demonstrated detectable Measles-specific IgG antibody titers approximately three weeks after the second administration (resulting in a mean antibody titre of 1.18). Approximately three weeks after the third administration and two weeks after the fourth administration, only a single animal (i.e. mouse C1) was found to be positive for Measles-specific IgG antibody titers.

In case of the group administered once on Day 0 with Measles vaccine Merieux®, an increase in the Measles-specific IgG response was detected 5 weeks after the administration in one out of two animals that have determined to be positive for such a response after approx. 3 weeks (the second animal showed similar levels at this time point). Approximately 9 or 11 weeks after administration of the commercially available Measles vaccine, the specific antibody titers were either decreasing to lower levels or dropping below the detection limit in mice F5 or F4, respectively. The three other mice from this group did not show a Measles-specific IgG response at any time. In case of the group administered once on Day 63 with 10⁸ TCID₅₀ MVA-mBN85B, a substantial Measles-specific IgG response was determined in all mice 14 days after the administration resulting in a mean Log₁₀ antibody titre of 3.88.

In summary, a good Measles-specific IgG antibody response was determined 20 days after a single subcutaneous administration with 10⁷ or 10⁸ TCID₅₀ MVA-mBN85B that was boosted by a second administration of the vaccine. With the higher dose, the IgG response could be further increased and a substantial antibody response was already determined 14 days after a first s.c. administration. In contrast to these two doses of MVA-mBN85B, single administration of the commercially available Measles vaccine Merieux® resulted only in partial induction of IgG responses in BALB/c mice and the antibody titers were substantially lower.

### The Cellular Immune Response by ELISpot Assay

Aside from investigating the measles-specific humoral response induced by MVA-mBN85B, another aim of the study was to investigate whether this recombinant MVA-product is able to mount an N-protein specific cellular immune response. For this purpose, the spleens were collected either two weeks after the fourth s.c. administration of three tested doses of MVA-mBN85B (Groups 3 to 5), MVA-BN® (Group 2), and TBS (Group 1) or two weeks after a single s.c. administration of MVA-mBN85B (Group 7). Spleens were also collected 11 weeks after s.c. administration of Measles vaccine Merieux (Group 6) although the major focus of this group was to investigate the measles-specific humoral immune responses. As shown in Table 9, splenocytes from all mice or from mice vaccinated with MVA-products were able to release IFNγ upon stimulation with staphylococcus enterotoxin B (SEB) or MVA-BN®, respectively (In some of these two cases, the numbers of spot forming cells (SFC) were not properly countable by the Zeiss Imaging System since too much IFN-γ was released and are therefore underestimated).

As shown in Table 9, stimulation of splenocytes from MVA-mBN85B vaccinated BALB/c mice (Groups 3, 4, 5, and 7) with peptide 1: YPALGLHEF (SEQ ID NO:11) or peptide 2: YAMGVGVELEN (SEQ ID NO:12) resulted in substantial release of IFN-γ whereas in either TBS treated (Group 1) or MVA-BN vaccinated animals no such release was detected. In case of peptide 1, the mean values were approximately 84, 161, or 59 SFC/million splenocytes following four subcutaneous administrations of 10⁶, 10⁷, or 10⁸ TCID₅₀ of MVA-mBN85B.

In order to demonstrate that MVA-mBN85B is able to mount an N-protein specific cellular immune response, four peptides were selected based either on literature search, Peptide 2: YAMGVGVELEN (SEQ ID NO:12) or on the scoring rates obtained from an epitope prediction data base called SYFPEITHI, Peptide 1: YPALGLHEF (SEQ ID NO:11) with a score of 27 for H2-L^{d} molecules; Peptide 3: SYAMGVGVEL (SEQ ID NO:13) with a score of 25 for H2-K^{d} molecules; Peptide 4: TYIVEAGLA (SEQ ID NO:14) with a score of 23 for H2-K^{d} molecules). Peptide 1 and peptide 2 were able to stimulate the highest IFNγ release from splenocytes vaccinated with 10⁷ TCID₅₀ of MVA-mBN85B, whereas peptide 3 raised a lower IFNγ release and peptide 4 was unable to stimulate such a response. Re-evaluation of the selected peptides with another epitope prediction data base called PRED^{BALB/C} confirmed the high score for binding of peptide 1 to H2-L^{d} molecules, but also revealed a good score for MHC class II molecules (i.e. I-A^{d} and I-E^{d}). Thus, it cannot be excluded that stimulation of the whole splenocyte suspension with peptide 1 stimulates both CD8 and CD4 T cell responses. With respect to peptide 2, the PRED^{BALB/c} data base predicts a good score for binding of peptide YAMGVGVEL to H2-D^{d} molecules. However, a much higher score predicts binding of peptide AMGVGVELE to I-A^{d} molecules thereby arguing for a more CD4 than a CD8 T cell biased IFNγ release upon restimulation with peptide 2. This theoretical finding would be in contrast to the fact that Halassy et al. (Vaccine, 2006 (24), pages 185-194) claim peptide 2 to be an H2^{d} restricted epitope. For clarification of this issue additional experiments that include stimulation of isolated CD4 or CD8 T cells from vaccinated mice with peptide 2 would be required. According to PRED^{BALB/c} data base (and similar to peptide 2), cellular responses detected upon stimulation of the whole splenocytes suspension with peptide 3 might be due to CD4 or CD8 T cell response.

Table 9 shows mean numbers (±S.E.M.) of splenocytes specifically secreting IFN-γ upon stimulation. Following incubation of 5×10⁵ splenocytes/well with the indicated stimuli or medium control, the numbers of IFNγ secreting cells were determined and the spot forming cells (SFC) per million splenocytes calculated. Baseline IFN-γ release upon incubation with medium was subtracted. Mean values including at least a single underestimated individual value are indicated with an asterix (*).

TABLE 9
Numbers of IFNγ secreting splenocytes

| | | | | | | | | | | Group 4 | | |
|---|---|---|---|---|---|---|---|---|---|---|---|---|
| | | | | Group 2 | | | Group 3 | | | 10⁷ TCID₅₀ | | |
| | Group 1 | | | 10⁸ TCID₅₀ | | | 10⁶ TCID₅₀ | | | MVA-mBN85B | | |
| | TBS | | | MVA-BN ® | | | MVA-mBN85B (4x) | | | (4x) | | |
| | (Administrations on | | | (Administrations on | | | (Administrations on | | | (Administrations on | | |
| | Days 0, 21, 42, 63) | | | Days 0, 21, 42, 63) | | | Days 0, 21, 42, 63) | | | Days 0, 21, 42, 63) | | |
| | Group | | | Group | | | Group | | | Group | | |
| Restimulation | mean | S.E.M. | N | mean | S.E.M. | N | mean | S.E.M. | N | mean | S.E.M. | N |
| Peptide 1 | 1.4 | 0.5 | 5 | 0.8 | 0.4 | 5 | 84.3 | 29.0 | 4 | 161.4 | 36.3 | 5 |
| Peptide 2 | 2.0 | 0.8 | 5 | 3.4 | 0.9 | 5 | 73.0 | 25.0 | 4 | 166.0 | 35.5 | 5 |
| Peptide 3 | 3.2 | 2.0 | 5 | 3.6 | 1.4 | 5 | 20.3 | 8.9 | 4 | 51.6 | 21.3 | 5 |
| Peptide 4 | 2.6 | 1.2 | 5 | 2.0 | 0.6 | 5 | 4.8 | 2.6 | 4 | 2.8 | 0.9 | 5 |
| Peptide pool | 1.6 | 0.6 | 5 | 3.0 | 0.5 | 5 | 80.3 | 24.2 | 4 | 175.6 | 34.9 | 5 |
| MVA-BN ® | 2.0 | 0.7 | 5 | 29.8* | 19.0 | 5 | 81.3* | 48.8 | 4 | 55.4* | 15.4 | 5 |
| SEB | 76.8 | 16.1 | 5 | 247.8 | 12.6 | 5 | 215.3 | 40.8 | 4 | 252.2 | 37.6 | 5 |
| Medium | 1.8 | 1.0 | 5 | 2.8 | 1.4 | 5 | 7.3 | 3.4 | 4 | 3.6 | 1.4 | 5 |

| | Group 5 | | | Group 6 | | | Group 7 | | |
|---|---|---|---|---|---|---|---|---|---|
| | 10⁸ TCID₅₀ | | | Measles vaccine | | | 10⁸ TCID₅₀ | | |
| | MVA-mBN85B (4x) | | | Merieux ® | | | MVA-mBN85B (1x) | | |
| | (Administrations on | | | (Administration on | | | (Administration on | | |
| | Days 0, 21, 42, 63) | | | Day 0 only) | | | Day 63 only) | | |
| | Group | | | Group | | | Group | | |
| Restimulation | mean | S.E.M. | N | mean | S.E.M. | N | mean | S.E.M. | N |
| Peptide 1 | 58.8 | 25.6 | 5 | 3.6 | 1.2 | 5 | 56.0 | 5.7 | 5 |
| Peptide 2 | 72.8 | 25.3 | 5 | 4.8 | 2.0 | 5 | 33.6 | 4.6 | 5 |
| Peptide 3 | 24.8 | 7.9 | 5 | 0.4 | 0.4 | 5 | 2.0 | 0.5 | 5 |
| Peptide 4 | 14.6 | 12.4 | 5 | 1.8 | 0.6 | 5 | 3.6 | 0.7 | 5 |
| Peptide pool | 68.4 | 27.9 | 5 | 6.0 | 1.8 | 5 | 58.2 | 8.1 | 5 |
| MVA-BN ® | 24.4* | 9.3 | 5 | 3.0 | 1.9 | 5 | 123.6* | 63.2 | 5 |
| SEB | 78.0 | 30.7 | 5 | 81.6 | 20.5 | 5 | 98.8* | 48.9 | 5 |
| Medium | 11.6 | 6.3 | 5 | 1.6 | 0.7 | 5 | 2.6 | 0.7 | 5 |

Stimulation of splenocytes with peptide 1 resulted in mean values of approximately 53 when mice were s.c. administered only once. A similar pattern was determined following stimulation either with peptide 2 or with a pool of the four peptides. Stimulation of the splenocytes with peptide 3 revealed a similar pattern, however, on a lower level: The highest mean value was determined in the group administered four times with 10⁷ TCID₅₀ of MVA-mBN85B. Peptide 4 did not stimulate release of IFN-γ at all. Furthermore, no measles N-protein-specific IFNγ release was determined from splenocytes when stimulated with specific peptides 11 weeks following s.c. administration of Measles vaccine Merieux®.

In summary, Measles N-protein specific cellular immune responses were determined 14 days after the last subcutaneous administration of MVA-mBN85B indirectly demonstrating protein expression in vivo. Following four s.c. administrations, the highest specific response was determined following vaccination with 10⁷ TCID₅₀ of MVA-mBN85B. The specific cellular immune response was in a similar range when MVA-mBN85B was administered s.c. either once or four times.

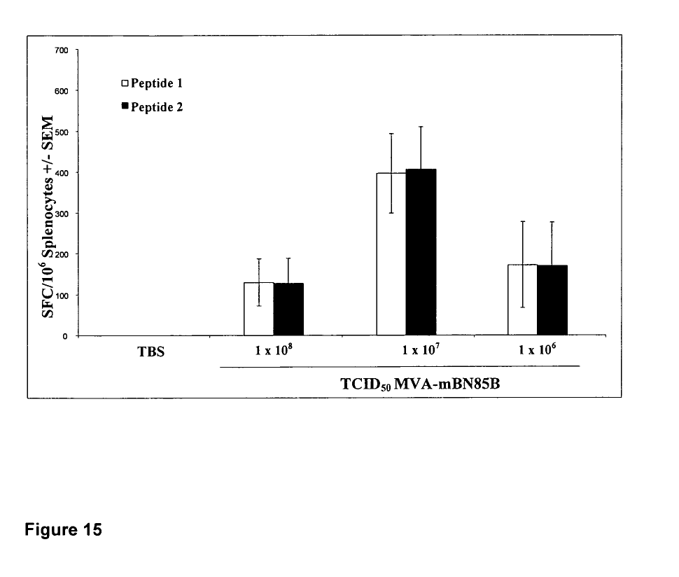

BN's measles vaccine MVA-mBN85B not only induces antibody responses as shown above, but also elicits T cell responses (FIG. 15). N-specific T cells were detected by their IFN-γ production in ELISpot assays at the end of each study: 14 days after the fourth and 14 days after the second administration (Days 77 and 35, respectively). In both studies, immunization with 1×10⁷ TCID₅₀ MVA-mBN85B induced the strongest T cell response (FIG. 15).

### Comparison of the Immunogenicity of MVA-mBN85B to Measles Vaccine Merieux® in Adult Mice

Another mouse study investigated humoral and cellular immune responses induced by MVA-mBN85B in comparison to the licensed measles vaccine Merieux®. The study was designed as described in Table 10. In order to compare T cell responses after one or two immunizations with MVA-mBN85B or with the commercial measles vaccine Merieux® under the same conditions (i.e. 14 days after the last immunization), some mice were immunized on Day 0 (and Day 21 in case of MVA-mBN85B), whereas others were immunized on Day 21 only.

TABLE 10
Summary of Study Design

| Test or Reference Item Administration | | | Bleed | Necropsy |
|---|---|---|---|---|
| | Dose | Schedule | Schedule | Schedule |
| Name | (TCID₅₀)^{a} | (Day*) | (Day*) | (Day*) |
| TBS | — | 0, 21 | −1, 14, 20, 28, 35 | 35 |
| MVA-mBN85B | 1 × 10⁸ | 0, 21 | −1, 14, 20, 28, 35 | 35 |
| Measles vaccine | ≧1 × 10³ | 0 | −1, 14, 20, 28, 35 | 35 |
| Merieux ® | | | | |
| Measles vaccine | ≧1 × 10³ | 21 | −1, 14, 20, 28, 35 | 35 |
| Merieux ® | | | | |
| MVA-mBN85B | 1 × 10⁸ | 21 | −1, 14, 20, 28, 35 | 35 |

*Relative to the first immunization.
^{a}Measles Vaccine Merieux dose used was the recommended human dose

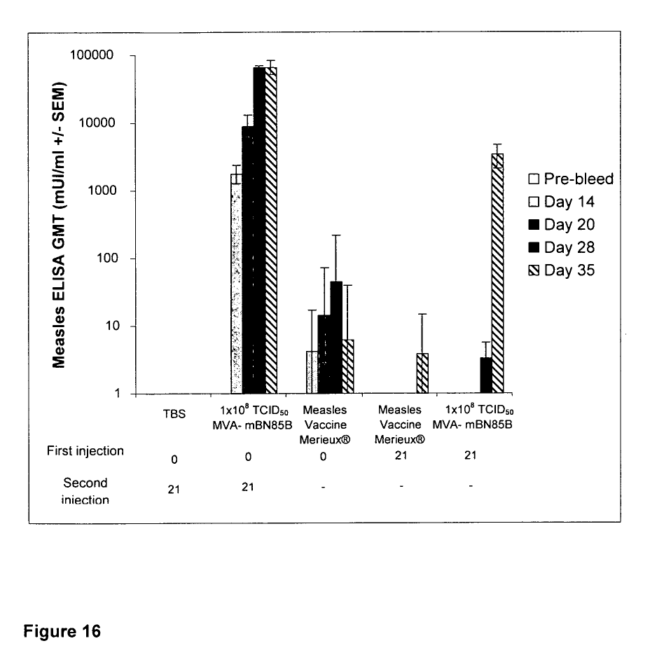

MVA-mBN85B was able to induce a good humoral immune response as early as 14 days after a single immunization (FIG. 16). This response increased with time (Day 20 post immunization) and could be boosted by a second vaccination. Merieux® required more time to elicit antibody responses, which slowly increased with time but did not reach high titers. Instead, titers decreased again three weeks after vaccination.

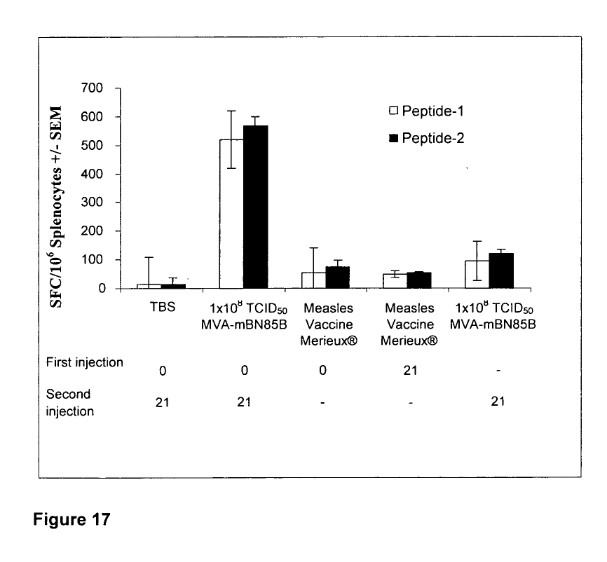

Results of the IFN-γ ELISpot assay on Day 35 confirm the ability of MVA-mBN85B to induce T cell responses (FIG. 17). Furthermore, the comparison of the two groups immunized once or twice with MVA-mBN85B showed that the T cell response could be boosted by more than 5-fold by a second immunization. Similar to antibody responses, MVA-mBN85B induced a much stronger T cell response in these mice than the measles vaccine Merieux®.

### Immunological Efficacy of MVA-mBN85B in Juvenile Rats

A major drawback of current measles vaccines is their lack of efficacy in children below 9 months of age. In order to gain insight into the feasibility of potential newborn and child vaccinations for clinical trials, immunogenicity of MVA-mBN85B was investigated in juvenile rats as well as neonatal and juvenile mice. The role of age and thereby the not-fully developed immune system of the animals in the induction of immunity was evaluated.

Sera samples taken from the juvenile rat study were evaluated to determine the humoral immune response following three vaccinations (s.c.) with MVA-mBN85B.

Sera were collected from rats on Day 34 after two immunizations and on Day 62 after three vaccinations, and antibody responses were measured by ELISA after repeated vaccinations using two different doses (1×10⁷ and 1×10⁸ TCID₅₀).

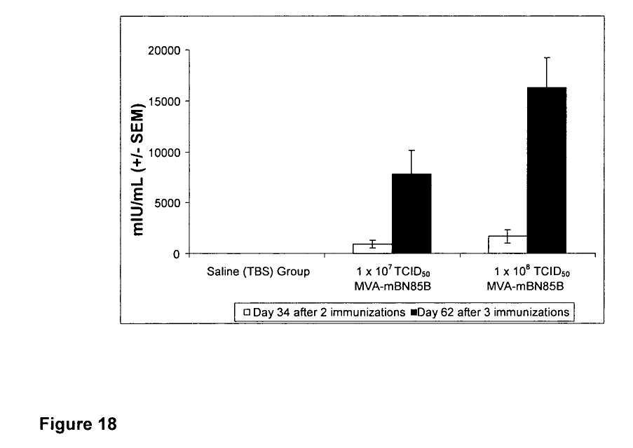

As illustrated in FIG. 18, there was a dose effect in the induction of measles-specific antibodies, with the highest MVA-mBN85B dose inducing mean titers about twice as high as the lower dose (932 mIU/ml ±367 SEM and 1,676 mIU/ml ±679 SEM for the 1×10⁷ and 1×10⁸ TCID₅₀ group, respectively). Following the third vaccination there was a clear boost effect with titers of 7,838 mIU/ml ±2,242 SEM and 16,281 mIU/ml ±2,952 SEM for animals treated with 1×10⁷ and 1×10⁸ TCID₅₀ MVA-mBN85B. All vaccinated animals (n=10) had seroconverted by that time.

### Immunogical Studies in Newborn and Juvenile Mice

A study was performed using 7 day old mice to model the partially developed immune system of a full-term human baby. Mice were immunized with two different doses of 1×10⁷ and 1×10⁸ TCID₅₀ MVA-mBN85B on Day 7 and/or Day 21, as described in Table 11.

TABLE 11
Summary of Study Design

| Test or Reference Item Administration | | | Bleed | Necropsy |
|---|---|---|---|---|
| | Dose | Schedule | Schedule | Schedule |
| Name | (TCID₅₀) | (Day*) | (Day*) | (Day*) |
| TBS | — | 7, 21 | °, 35, 49, 63 | 35, 63 |
| MVA-mBN85B | 1 × 10⁸ | 7, 21 | °, 35, 49, 63 | 35, 63 |
| MVA-mBN85B | 1 × 10⁸ | 7 | 20, 35, 49, 63 | 35, 63 |
| MVA-mBN85B | 1 × 10⁸ | 21 | 20, 35, 49, 63 | 35, 63 |
| MVA-mBN85B | 1 × 10⁷ | 7, 21 | °, 35, 49, 63 | 35, 63 |
| MVA-BN ® | 1 × 10⁸ | 7, 21 | °, 35, 49, 63 | 35, 63 |

*Relative to the day of birth,
° On Day 20, bleed was done only for the third and fourth group and results of group one and two was extrapolated.

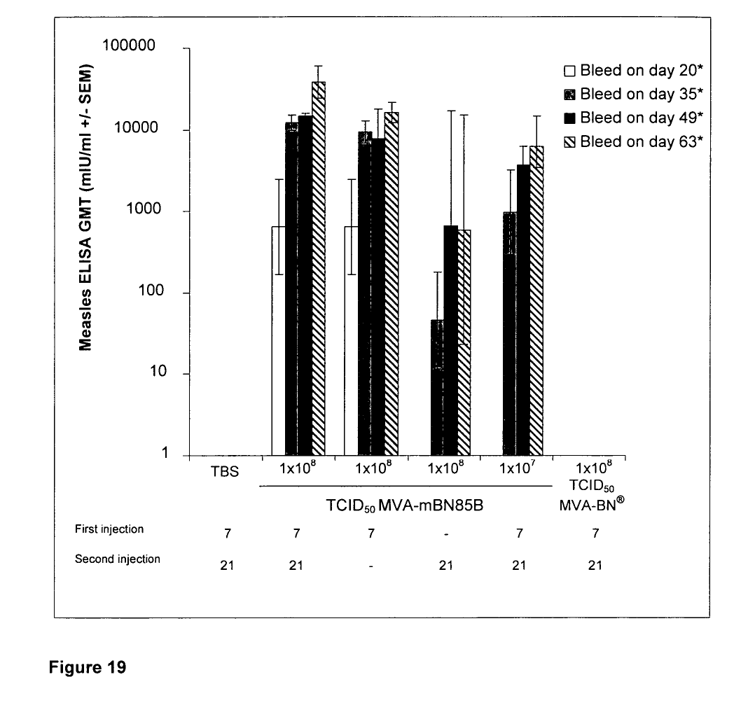

As depicted in FIG. 19, mice immunized on Day 7 with 1×10⁸ TCID₅₀ MVA-mBN85B developed similar humoral immune responses (100% seroconversion with titers from 2,624 to 21,322 mIU/ml) than those observed for the same dose in adults. In the previously described experiment conducted in adults, the increase in measles-specific antibody titers before and after the second immunization was interpreted as a boost effect. However, a similar increase was observed when mice were immunized only once on Day 7 with 1×10⁸ TCID₅₀ MVA-mBN85B, rather than twice on Days 7 and 21. This observation indicates that a single immunization might be sufficient to achieve a potentially protective antibody response.

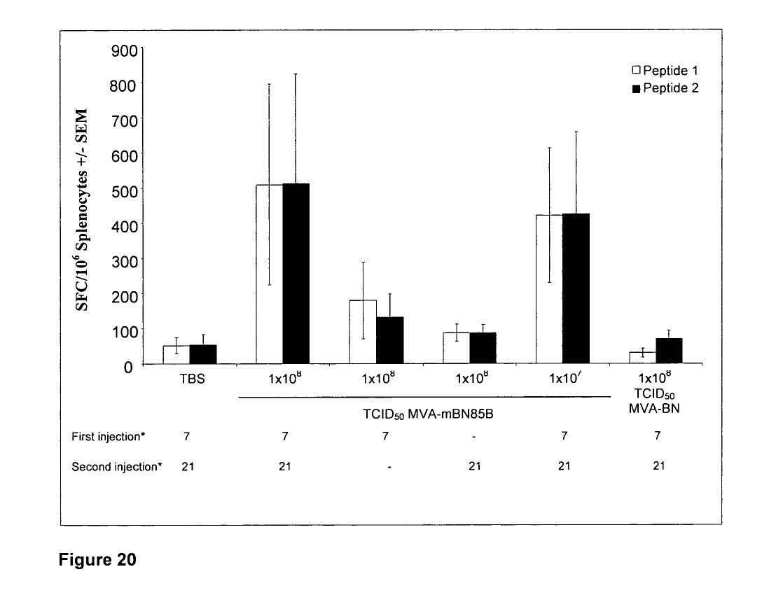

In addition to antibody responses, juvenile mice immunized with MVA-mBN85B also demonstrated a T cell response. An optimal T cell response was observed, when mice were immunized twice, as previously described for adult animals. There was no difference between mice immunized twice with 1×10⁸ TCID₅₀ or 1×10⁷ TCID₅₀ MVA-mBN85B (see FIG. 20).

The immune responses induced by immunization of newborn mice on the day of birth was compared to immunization of 7 days old mice. Newborn mice are considered equivalent to a premature human baby in terms of the development of their immune system. A study was designed as shown in Table 12.

TABLE 12
Summary of Study Design

| Test or Reference Item Administration | | | Bleed | Necropsy |
|---|---|---|---|---|
| | Dose | Schedule | Schedule | Schedule |
| Name | (TCID₅₀) | (Day*) | (Day*) | (Day*) |
| TBS | — | 7, 21 | 20, 35, 49, 63, 84, | 35 |
| MVA- | 1 × 10⁸ | 7, 21 | 105, 126, 147, 168, | |
| mBN85B | | | | |
| MVA- | 1 × 10⁸ | 0, 21 | 189 | |
| mBN85B | | | | |

*Relative to the day of birth.

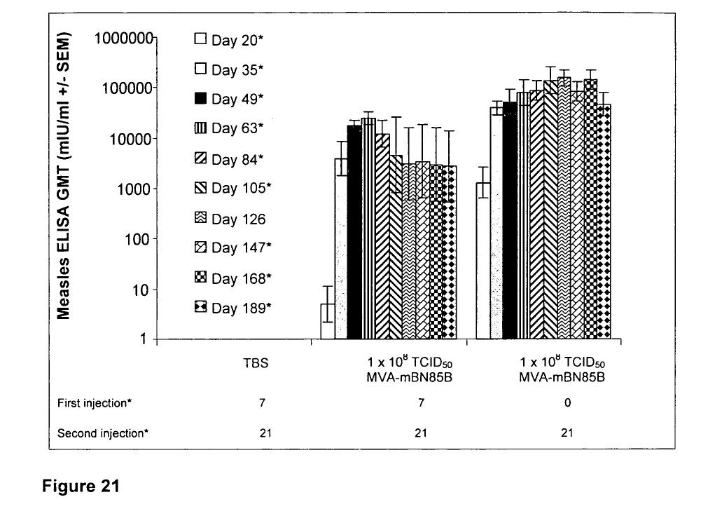

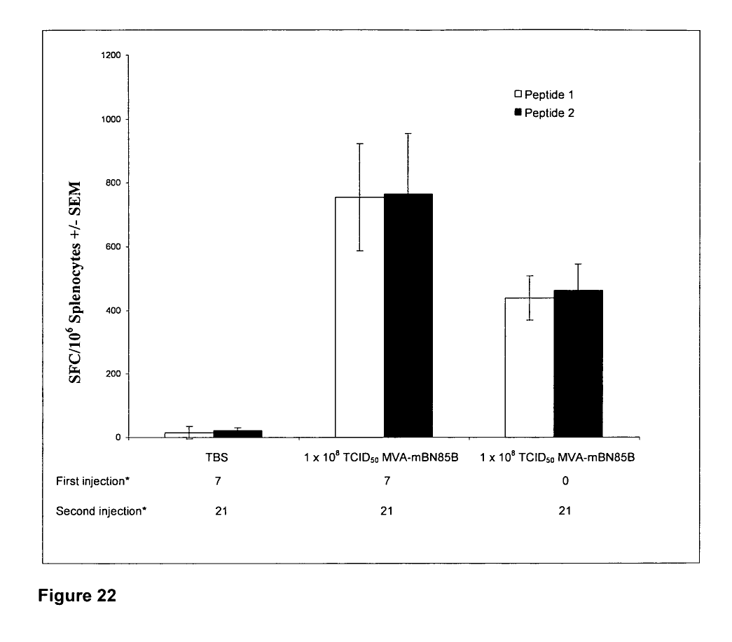

The results of the humoral (FIG. 21) and cellular (FIG. 22) responses show no major differences between newborn and 7 day old mice. The seroconversion rate of 100% starting on Day 35 and magnitudes of maximal 24,530 mIU/ml when immunized on Day 7 and 154,497 mIU/ml when immunized on Day 0 are similar to those observed in adult mice. High titers were maintained up to 24 weeks after vaccination, indicating a long lasting immunity.

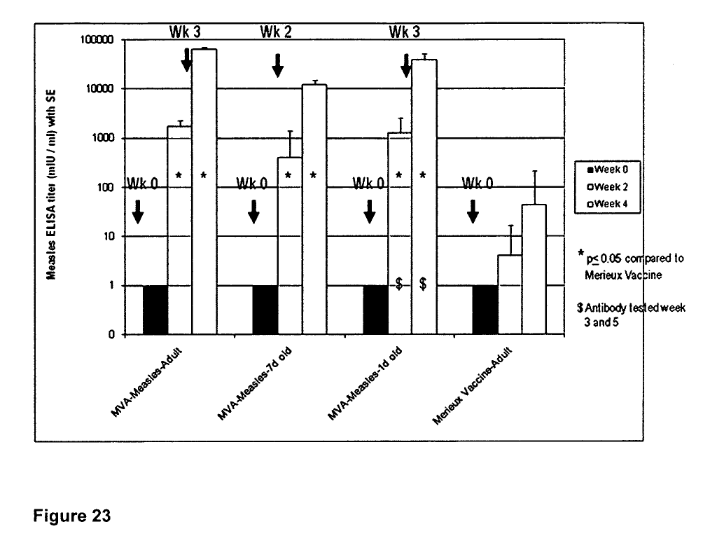

The immune response to MVA-mBN85B in adult, 7 day old, and 1 day old mice was compared to the immune response to the commercial measles vaccine, Rouvax, in adult mice. Subjects received either 2 doses of MVA-mBN85B (1×10⁸ TCID₅₀) or the recommended dose of Rouvax. Anti-measles antibodies were measured pre-vaccination and at 2 and 4 weeks after vaccination (FIG. 23). A much higher humoral immune response was seen with MVA-mBN85B in all mice, regardless of age, as compared to the immune response with Rouvax in adult mice. Thus, MVA-mBN85B induces a more robust immune response to the measles virus and is a superior vaccine for the measles virus as compared to Rouvax in adults, newborns, and juveniles.

### Immunogical Studies in Newborn after a Single Immunization

The immune responses induced by a single immunization of newborn mice on the day of birth or single immunization of 7 days old mice was compared to mice that were boosted on day 21. Newborn mice are considered equivalent to a premature human baby in terms of the development of their immune system. A study was designed as shown in Table 13.

TABLE 13
Summary of Study Design

| Test or Reference Item Administration | | | Bleed |
|---|---|---|---|
| | Dose | Schedule | Schedule |
| Name | (TCID₅₀) | (Day*) | (Day*) |
| TBS | — | 0, 21 | 20, 35, 49, 63, 84, 105, 126, |
| MVA-mBN85B | 1 × 10⁸ | 0, 21 | 147, 168, 189 |
| MVA-mBN85B | 1 × 10⁸ | 7, 21 | |
| MVA-mBN85B | 1 × 10⁸ | 0 | |
| MVA-mBN85B | 1 × 10⁸ | 7 | |

*Relative to the day of birth.

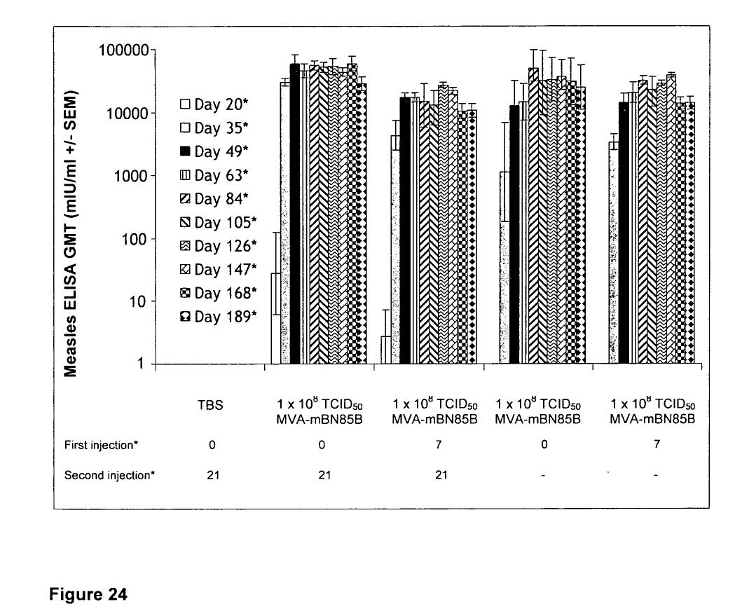

The results of the humoral responses (FIG. 24) show no major differences between newborn or 7 day old mice that received a single dose compared to the groups boosted on day 21. For the groups that were immunized twice, the seroconversion rate of 100% starting on Day 35 and magnitudes of maximal 27,196 mIU/ml when immunized on Day 7 and 59,858 mIU/ml when immunized on Day 0 are similar to the results obtained previously (FIG. 22). The seroconversion rate of 100% starting on Day 35 and magnitudes of maximal 38,939 mIU/ml when immunized on Day 7 only or the seroconversion rate of 100% on Day 49 and magnitudes of maximal 49,918 mIU/ml when immunized on Day 0 only are similar to those observed in adult mice or in newborn/juvenile mice immunized twice. High titers were maintained up to 27 weeks after for the group immunized the day of birth vaccination, indicating a long lasting immunity even after a single vaccination of newborn mice.

### Clinical Experience with MVA-mBN85B

To date, more than 2,700 individuals have already been vaccinated with MVA-BN®-based vaccines. In addition, the safety of MVA-based recombinant vaccines like MVA-mBN85B has been demonstrated in more than 250 immunocompromised subjects, i.e. at-risk populations like subjects with HIV infection or patients with AD. MVA-based vaccines were used at doses up to five times higher than those typically used when MVA-BN® is administered alone. All vaccines in these studies seemed to be safe and well tolerated.

Clinical trials were conducted according to international ethical and scientific quality standards (ICH-GCP), in compliance with the Declaration of Helsinki and the national drug laws applicable at the time of conduct.

One clinical study in 30 healthy, 18 to 32 year old adults has been performed using MVA-mBN85B as the investigational medicinal product. The study subjects received two vaccinations of 1×10⁸TCID₅₀ MVA-mBN85B four weeks apart. Final data show that no Severe Adverse Event (SAE) was recorded and no unusual safety issues were raised after the two vaccinations. There were no drop-outs until day 56. For the final follow-up visit at day 210 one subject was lost to follow up. The local reactogenicity was comparable to other MVA-B0-based vaccines. At baseline, 28 subjects were measles ELISA seropositive and only two had a titer below the detection limit. Table 14 shows data about the immune response of MVA-mBN85B in healthy, young adults.

TABLE 14
Data of the Immune Response after Two Vaccinations Four
Weeks Apart with MVA-mBN85B in Healthy, Young Subjects.

| | | | | | | | LL | |
|---|---|---|---|---|---|---|---|---|
| Day | Visit | n | GMT | LL CI | UL CI | SC % | SC % | UL SC % |
| 0 | 1 | 30 | 778 | 473 | 1278 | NA | NA | NA |
| 14 | 2 | 30 | 8420 | 6427 | 11030 | 96.7 | 83.3 | 99.4 |
| 28 | 3 | 30 | 6413 | 4825 | 8523 | 93.3 | 78.7 | 98.2 |
| 42 | 4 | 30 | 6286 | 4720 | 8371 | 96.7 | 83.3 | 99.4 |
| 56 | 5 | 30 | 4685 | 3549 | 6184 | 86.7 | 70.3 | 94.7 |
| 210 | FU | 29* | 1644 | 1093 | 2473 | 53.3 | 36.1 | 69.8 |

GMT = geometric mean titers in mIU/ml;
SC % = seroconversion rate and is defined as a titer above the assay cut-off of 150, if the subject was seronegative at Baseline. If a subject showed a Baseline titer, a two-fold increase compared to the Baseline titer was necessary to count as seroconversion;
LL and UL CI = lower and upper limit 95% confidence interval;
NA = not applicable;
FU = follow up;
*one subject was lost to follow up at day 210.

MVA-mBN85B induced a strong booster response in 28 subjects with detectable baseline measles titers. Also the two subjects with no detectable titer at baseline showed high titers (>2000 mIU/ml) after the first vaccination. It is not clear, whether they had not been vaccinated against measles or did not have detectable titers anymore. These data suggest that MVA-mBN85B is able to induce a strong measles-specific immune response confirming the encouraging preclinical data in mice and rats.

The most prevalent solicited general adverse effects after the 1^{st} vaccination were muscle pain (11 subjects, 36.7%), headache (10 subjects, 33.3%), and fatigue (9 subjects, 30.0%). These were all classified as Grade 1 AEs. The most prevalent solicited general AE after the 2^{nd} vaccination was Grade 1 fatigue (9 subjects, 30.0%). Both muscle pain and headache were more prevalent in the 7 days after the 1^{st} vaccination (muscle pain [11 subjects, 36.7%] and headache [10 subjects, 33.3%]) than in the 7 days after the 2^{nd} vaccination (muscle pain [5 subjects, 16.7%] and headache [5 subjects, 16.7%]).

The most prevalent solicited local AE after the 1^{st} vaccination was pain (Grade 1: 14 subjects, 46.7%; Grade 2: 14 subjects, 46.7%). Pain was also the most prevalent solicited local AE after the 2^{nd} vaccination (19 subjects, 63.3%). These were all Grade 1 AEs.

There were five solicited local AEs of Grade 3 intensity:
Subject 05 experienced pain, which was severe on the 1^{st}, 5^{th} and 6^{th} days after the 1^{st}vaccination, moderate on the 2^{nd} and 3^{rd} days after the 1^{st} vaccination, and mild on the day of the 1^{st} vaccination, 4^{th} and 7^{th} days after the 1^{st} vaccination.
Subject 09 experienced redness on the day of the 2^{nd} vaccination (150 mm), 1^{st} day (150 mm), 2^{nd} day (110 mm) and 3^{rd} day (100 mm) after the 2^{nd} vaccination, and induration on the day of the 2^{nd} vaccination (120 mm), 1^{st} day (50 mm) after the 2^{nd} vaccination.
Subject 11 experienced swelling on the day of the 1^{st} vaccination (110 mm) and for the next 7 days thereafter (ranging from 110 mm on the 1^{st} day to 67 mm on the 7^{th}). The swelling persisted for another 4 days, at which time it measured 60 mm.
Subject 21 experienced pain, which was severe on the day of the 1^{st} vaccination and the 1^{st} day after the 1^{st} vaccination, moderate on the 2^{nd} day after the 1^{st} vaccination, and mild on the 3^{rd}, 4^{th}, 5^{th} and 6^{th} days after the 1^{st} vaccination.

This spectrum of local AEs within the first week after vaccination is comparable to other MVA-BN®-based vaccines and is seen with many other modern vaccines. It is a sign of a robust immune response.

Headache was the most common unsolicited AE (seven mentions) experienced after the 1st vaccination. Very few unsolicited AEs were experienced after the 2nd vaccination. Two subjects experienced unsolicited AEs≧Grade 3. Subject 05 experienced toothache 17 days after the 1st vaccination. The AE was severe in intensity and not related to the study vaccine. It resolved after 4 days. Subject 17 experienced a headache 27 days after the 2nd vaccination that lasted 1 day. The AE was severe in intensity and not related to study vaccine. The spectrum of unsolicited AEs does not indicate any unusual safety trend. These AEs are common AEs for vaccine studies. In summary, MVA-mBN85B showed a very strong immune response in this study and was safe and well tolerated.

Humoral response against measles was tested by a measles-specific Plaques Reduction Neutralization Test (PRNT). In the PRNT test 97% of the subjects (29/30) had detectable measles neutralizing antibodies at baseline which closely correlate with the measles ELISA results. The Spearman correlation between the measles-specific ELISA and measles-specific PRNT results calculated from all recorded points during visits 1 through the follow up visit (day 0 to day 210) was 0.86 at p value of <0.0001. Summary of the PRNT results has been shown in Table 15.

TABLE 15
Data of the Measles-specific PRNT Titers after Two
Vaccinations Four Weeks Apart with MVA-mBN85B in Healthy,
Young Subjects.

| | | Geometric | | | | 95% CI |
|---|---|---|---|---|---|---|
| Day | N | Mean | SD | Minimum | Maximum | for geometric mean |
| 0 | 30 | 128 | 186.34 | 8 | 1163 | (86.1; 190.9) |
| 14 | 30 | 789 | 1082.13 | 36 | 6371 | (537.1; 1158.1) |
| 28 | 30 | 520 | 747.83 | 29 | 4036 | (350.4; 772.5) |
| 42 | 30 | 454 | 499.41 | 30 | 1758 | (325.7; 633.2) |
| 56 | 30 | 346 | 405.54 | 29 | 1417 | (244.4; 489.5) |
| 210 | 29 | 167 | 186.13 | 18 | 1139 | (118.3; 234.7) |

N = number of subjects;
SD = standard deviation;
CI = confidence interval

The humoral response to Vaccinia induced by MVA-mBN85B was tested by a Vaccinia-specific ELISA. The results of the Vaccinia-specific ELISA are shown in Table 16.

TABLE 16
Data of the Vaccinia-specific ELISA Titers after Two
Vaccinations Four Weeks Apart with MVA-mBN85B in
Healthy, Young Subjects.

| | | Geometric | | | | 95% CI |
|---|---|---|---|---|---|---|
| Day | n | Mean | SD | Minimum | Maximum | for geometric mean |
| 0 | 30 | 3.7 | 21.07 | 1.0 | 50.0 | (1.8; 7.4) |
| 14 | 30 | 86.8 | 209.88 | 1.0 | 810.0 | (51.7; 145.7) |
| 28 | 30 | 136.9 | 200.20 | 1.0 | 442.0 | (91.8; 204.1) |
| 42 | 30 | 644.4 | 725.14 | 100.0 | 6429.0 | (459.7; 903.3) |
| 56 | 30 | 397.1 | 294.38 | 100.0 | 1717.0 | (310.1; 508.4) |
| 210 | 29 | 42.4 | 94.23 | 1.0 | 100.0 | (25.5; 70.4) |

n = number of subjects;
SD = standard deviation;
CI = confidence interval

The results above show that the MVA-measles vaccine induces antibodies against measles and smallpox simultaneously. The peak of anti-vaccinia antibodies was reached 14 days after dose 2, all subjects were seroconverted. MVA-mBN85B induced a strong measles boost response in the measles experienced subjects and induced strong antibody response against Vaccinia.

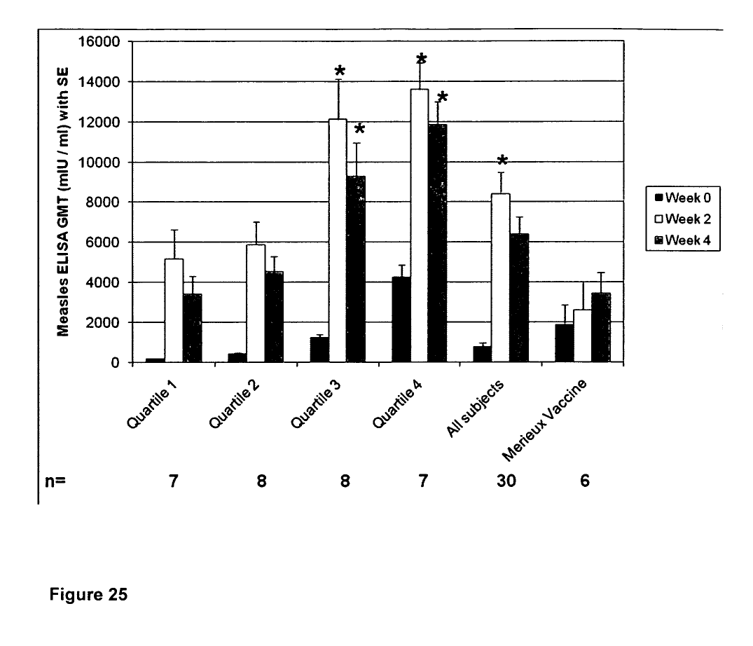

MVA-mBN85B was compared to the commercial measles vaccine, Rouvax, in human subjects. Subjects received either 1 dose of MVA-mBN85B (1×10⁸ TCID50) or the recommended dose of Rouvax. Anti-measles antibodies were measured pre-vaccination and at 2 and 4 weeks after vaccination (FIG. 25). A 275% better response was seen with MVA-mBN85B compared to Rouvax. Thus, MVA-mBN85B induces a more robust immune response to the measles virus and is a superior vaccine for the measles virus as compared to Rouvax.

1-29. (canceled)

30. A Highly Attenuated Modified Vaccinia Virus Ankara (HA-MVA) virus encoding the hemagglutinin protein (H), fusion protein (F), and nucleoprotein (N) of the measles virus;
wherein administration of the HA-MVA encoding the H, F, and N of the measles virus to mice induces a stronger humoral and cellular immune response against the measles virus than that induced by a single immunization with Rouvax vaccine (Schwartz strain 1000 TCID₅₀).

31. The HA-MVA virus of claim 30, wherein the HA-MVA virus is a derivative of MVA-BN.

32. The HA-MVA virus of claim 30, wherein the HA-MVA virus has a virus amplification ratio at least three fold less than MVA-575 in Hela cells and HaCaT cell lines.

33. The HA-MVA virus of claim 30, wherein the HA-MVA virus has an amplification ratio of greater than 500 in CEF cells.

34. The HA-MVA virus of claim 30, wherein the expression of the H, F, and N proteins of the measles virus is under the control of cowpox virus ATI promoters.

35. The HA-MVA virus of claim 30, wherein the H, F, and N proteins of the measles virus are inserted into intergenic regions of the HA-MVA.

36. The HA-MVA virus of claim 35, wherein the H, F, and N proteins of the measles virus are inserted into intergenic regions IGR 64/65, IGR07/08, and IGR 44/45 of the HA-MVA.

37. A cell comprising the HA-MVA virus of claim 30.

38. A vaccine comprising a dose of 10⁷ TCID₅₀ to 10⁸ TCID₅₀ of the HA-MVA virus of claim 30.

39. The vaccine of claim 38, comprising a dose of 10⁷ TCID₅₀ of the HA-MVA virus.

40. The vaccine of claim 38, comprising a dose of 10⁸ TCID₅₀ of the HA-MVA virus.

41. A kit comprising one or multiple vials of the HA-MVA virus of claim 30 and instructions for the administration of the virus to a subject.

42. A method for immunizing a human comprising administering a dosage of 10⁷ to 10⁸ TCID₅₀ of the HA-MVA of claim 30 to a human subject.

43. The method of claim 42, wherein the human subject is an adult.

44. The method of claim 42, wherein the human subject's age is less than 12 months.

45. The method of claim 44, wherein the human subject's age is less than 9 months.

46. The method of claim 45, wherein the human subject's age is less than 6 months.

47. The method of claim 46, wherein the human subject's age is less than 3 months.

48. The method of claim 42, wherein the HA-MVA virus is administered in a first (priming) and second (boosting) administration.

49. The method of claim 42, wherein a single immunization with the HA-MVA virus induces a Measles ELISA geometric mean titer at least 2-fold greater that that induced by a single immunization with Rouvax vaccine (Schwartz strain 1000 TCID₅₀) in humans.


SEQUENCE LISTING (14 sequences)

SEQ ID NO: 1 - DNA, 1854 bp, Measles virus
atgtcaccgcaacgagaccggataaatgccttctacaaagataacccttatcccaagggaagtaggatagttattaacagagaacatcttatgattgacagaccctatgttttgctggctgttctgttcgtcatgtttctgagcttgatcgggttgctggccattgcaggcattagacttcatcgggcagccatctacaccgcggagatccataaaagcctcagtaccaatctagatgtgactaactccatcgagcatcaggtcaaggacgtgctgacaccactctttaaaatcatcggggatgaagtgggcctgagaacacctcagagattcactgacctagtgaaattcatctctgacaagattaaattccttaatccggatagggagtacgacttcagagatctcacttggtgcattaacccgccagagagaatcaaactggattatgatcaatactgtgcagatgtagctgctgaagagctcatgaatgcattggtgaactcaactctactggagaccagaacaaccaatcagttcctagctgtctcaaagggaaactgctcagggcccactacaatcagaggtcaattctcaaacatgtcgctgtccttgttggacttgtacttaggtcgaggttacaatgtgtcatctatagtcactatgacatcccagggaatgtatgggggaacctacctagtggaaaagcctaatctgaacagcaaagggtcagagttgtcacaactgagcatgtaccgagtgtttgaagtaggtgttatcagaaacccgggtttgggggctccggtgttccatatgacaaattattttgagcaaccagtcagtaatggtctcggcaactgtatggtggctttgggggagctcaaactcgcagccctttgtcacggggacgattctatcacaattccctatcagggatcagggaaaggtgtcagcttccagctcgtcaagctgggtgtctggaaatccccaaccgacatgcaatcctgggtctccttatcaacggatgatccagcggtagacaggctttacctctcatctcacagaggtgtcatcgctgacaatcaagcaaaatgggctgtcccgacaacacgaacagatgacaagctgcgaatggagacatgcttccagcaggcgtgtaaaggtaaaatccaagcactctgcgagaatcccgagtgggcaccattgaaggataacaggattccttcatacggggtcctgtctgttgatctgagtctgacggttgagcttaaaatcaaaattgcttcgggattcgggccattgatcacacacggctcagggatggacctatacaaatccaaccgcaacaacgtgtattggctgactatcccgccaatgaggaatctagccttaggcgtaatcaacacattggagtggataccgagattcaaggttagtcccaacctcttcactgtcccaattaaggaagcaggcgaagactgccatgccccaacatacctacctgcggaagtggacggtgatgtcaaactcagttccaacctggtgatcctacctggtcaagatctccaatatgttttggcaacctacgatacttccagggttgagcatgctgtggtttattacgtttacagcccaagccgctcattttcttacttttatccttttaggttgcctataaagggggtcccaatcgaattacaagtggaatgcttcacatgggaccaaaaactctggtgccgtcacttctgtgtgcttgcggactcagaatccggtggacttatcactcactctgggatggtaggcatgggagtcagctgcacagccacccgggaagatggaaccaaacgcagatag

SEQ ID NO: 2 - DNA, 1653 bp, Measles virus
atgggtctcaaggtggacgtctttgccatattcatggcagtactgttaactctccaaacacccgccggtcaaatccattggggcaatctctctaagataggtgtagtaggaataggaagtgcaagctacaaagttatgactcgttccagccatcaatcattagtcataaaattaatgcccaatataactctcctcaataactgcacgagggtagagattgcagaatacaggagactactaagaacagttttggaaccaattagagatgcacttaatgcaatgacccagaacataaggccggttcagagcgtagcttcaagtaggagacacaagagatttgcgggagtagtcctggcaggtgcggccctaggtgttgccacagctgctcagataacagccggcattgcgcttcaccagtccatgctgaactctcaggccatcgacaatctgagagcgagcctggaaactactaatcaggcaattgaggcaatcagacaagcagggcaggagatgatattggctgttcagggtgtccaagactacatcaataatgagctgataccgtctatgaaccaactatcttgtgatttaatcggtcagaagctcgggctcaaattgctcagatactatacagaaatcctgtcattatttggccccagcctacgggaccccatatctgcggagatatctatccaggctttgagctatgcacttggaggagatatcaataaggtgttagaaaagctcggatacagtggaggcgatttactaggcatcctagagagcagaggaataaaggctcggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcatgtactttcatgccagaggggactgtgtgcagccaaaatgccttgtacccgatgagtcctctgctccaagaatgcctccgagggtccaccaagtcctgtgctcgtacactcgtatccgggtcttttgggaaccggttcattttatcacaagggaacctaatagccaattgtgcatcaattctttgtaagtgttacacaacaggaacgatcattaatcaagaccctgacaagatcctaacatacattgctgccgatcgctgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgccaaattggaggatgccaaggaattgttggaatcatcggaccagatattgaggagtatgaaaggtttatcgagcactagcatagtctacatcctgattgcagtgtgtcttggagggttgatagggatccccactttaatatgttgctgcagggggcgttgtaacaaaaagggggaacaagttggtatgtcaagaccaggcctaaagcctgaccttacaggaacatcaaaatcctatgtaaggtcgctttga

SEQ ID NO: 3 - DNA, 1578 bp, Measles virus
atggccacacttttgaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtcaggttaattggaagcccggatgtgagcgggcccaaactaacaggggcactaataggtatattatccttatttgtggagtctccaggtcaattgattcagaggatcactgatgaccctgacgttagcataaggctgttagaggttgtccagagcgaccagtcacaatctggccttaccttcgcatcaagaggtactaatatggagtatgaggcggaccagtacttttcacatgatgatccaagtagtagtgatcaatccaggttcgggtggtttgagaacaaggaaatctcagatattgaagtgcaagaccctgagggcttcaacatgattctgggtaccatcctagctcaaatttgggtcttgctcgcaaaggcggttacggctcctgacacagcagctgattcggagctaagaaggtggatcaaatacacccaacaaagaagagtagttggtgaatttagattagagagaaaatggttggatgtggtgagaaacaggattgccgaggacctctccttacgccgattcatggtcgctctaatcctggatatcaagaggacacccgggaacaaacccaggattgctgaaatgatatgtgacattgatacatatatcgtagaggcaggattagccagttttatcctgactattaagtttggaatagaaactatgtatcctgctcttggactgcatgaatttgctggtgaattatccacacttgagtccttgatgaatctttaccagcaaatgggggaaactgcaccctatatggtaatcctggagaactcaattcagaacaagtttagcgcaggatcataccctttgctctggagctatgccatgggagtaggagtggaacttgaaaactccatgggaggtttgaacttcggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaggatcagtagagcagttggacccagacaagcccaagtgtcattcctacacggtgatcaaagtgagaatgagctgccgagattggggggcaaggaggacaggagggtcaaacagagccgaggagaagccggggagagccacagagaaaccgggcccagcagagcaagtgatgcgagagctgcccatcctccaaccggcacacccctagacattgacactgcatcggagttcagccaagatccgcaggacagtcgaaggtcagccgatgccctgcttaggctgcaagccatggcaggaatctcggaagaacaagactcagacacggacacccctagagtgtacaatgacagagatcttctagactag

SEQ ID NO: 4 - DNA, 30 bp, Cowpox virus
gttttgaataaaatttttttataataaatc

SEQ ID NO: 5 - DNA, 21 bp, Modified Vaccinia Virus Ankara
ctggataaatacgaggacgtg

SEQ ID NO: 6 - DNA, 20 bp, Modified Vaccinia Virus Ankara
gacaattatccgacgcaccg

SEQ ID NO: 7 - DNA, 20 bp, Modified Vaccinia Virus Ankara
caactctcttcttgattacc

SEQ ID NO: 8 - DNA, 19 bp, Modified Vaccinia Virus Ankara
cgatcaaagtcaatctatg

SEQ ID NO: 9 - DNA, 24 bp, Modified Vaccinia Virus Ankara
cgttagacaacacaccgacgatgg

SEQ ID NO: 10 - DNA, 21 bp, Modified Vaccinia Virus Ankara
cggatgaaaaatttttggaag

SEQ ID NO: 11 - protein, 9 aa, Measles virus
YPALGLHEF

SEQ ID NO: 12 - protein, 11 aa, Measles virus
YAMGVGVELEN

SEQ ID NO: 13 - protein, 10 aa, Measles virus
SYAMGVGVEL

SEQ ID NO: 14 - protein, 9 aa, Measles virus
TYIVEAGLA